(19) United States
(12) Patent Application Publication
(10) Pub. No.: US 2022/0122700 A1
(43) Pub. Date: Apr. 21, 2022

(54) Predictive Electronic Healthcare Record Systems and Methods for the Developing World

(71) Applicant: LiveBeyond, Thomazeau (HT)

(72) Inventor: David Vanderpool, Nashville, TN (US)

(21) Appl. No.: 17/075,567

(22) Filed: Oct. 20, 2020

(51) Int. Cl. G16H 10/60; G16H 50/20; G16H 50/70; G16H 80/00; G16H 50/80; G16H 40/67; G16H 40/20; G06N 20/00

(52) U.S. Cl. CPC G16H 10/60; G16H 50/20; G16H 50/70; G06N 20/00; G16H 50/80; G16H 40/67; G16H 40/20; G16H 80/00

(57) ABSTRACT

A unified and integrated medical data management tool system comprised of multiple application modules, capable of operating on multiple common operating systems and platforms and using readily available digital devices, for the management of Electronic Medical Records (EMR) so as improve multiple key healthcare metrics across an entire national healthcare system, being able to: improve the nationwide level of medical education; evaluate the efficiency and appropriateness of physician diagnosis and treatment decisions and a knowledgebase to provide suggested alternatives; facilitate electronic entry of data generated by medical devices; track epidemiological cases and data and forward such data to national and global health agencies; utilize a knowledgebase to facilitate the search for, and coordination with, medical specialists, and sources of medicines medical supplies; coordinate medical care in the event of natural disaster; and facilitate the collection and presentation of health data in applying for grant funding from agencies and foundations.

### BACKGROUND OF THE INVENTION

The invention relates to software for systematic medical decision-making, education, patient case review, health outcomes reporting, and epidemiological data gathering, analysis and reporting. More specifically, the present disclosure relates to computational methods utilizing multiple alternative operating systems and platforms, computing devices, medical devices and apparatus, and communications devices and systems, for clinical medicine decision-making, medical education, patient case reviews to improve individual and populational health outcomes, and for gathering and analyzing data on communicable disease that can be forwarded to national and international health agencies through the use of medical artificial intelligence-type software.

### BRIEF SUMMARY OF THE INVENTION

In summary, the invention is a unified and integrated Electronic Medical Records (EMR) national healthcare management tool system and method utilizing multiple technologies, including for example, telemedicine, artificial intelligence (AI) in the practice of medicine, e-learning, and video enabled software tools for interaction with medical devices, all purposed for improving multiple healthcare metrics across an entire national healthcare system.

In a preferred embodiment, the invention is a national healthcare data management tool system adapted for execution on a computer system coupled to a global computer network, in an architecture of a server in communication with a plurality of clients in a network, comprising at least one predictive artificial intelligence module for processing medical or health data received from one or more specialized computation modules, in consideration of patient data to identify a diagnosis and therapy for a patient, or in consideration of patient data from a population of patients to identify therapy for a diagnosis common to members of the patient population; a national medical information database; a gateway module configured to provide said artificial intelligence module with access to said database; and a communications module operable to enable any of said modules to send or receive data from any other module in said healthcare management tool system, and to send data to, or receive data from, a plurality of clients. This system can additionally comprise an education module operable to present medical educational content to one or more clients in said network; comprising a medical provider evaluation module operable to present an evaluation of appropriateness of a medical provider's diagnosis or treatment decisions and communicate recommendations to said provider in consideration of said evaluation of appropriateness; a communicable disease module operable to receive communicable disease data from one or more of said network clients, present said data to said artificial intelligence module, receive output data from said artificial intelligence module, and send output data on said communicable disease to one or more of said clients; a specialist referral module operable to assess a need for the participation of a specialty physician in the management of a patient's case and assist in the location of such a specialty physician, and assist in coordinating a patient examination by said specialty physician; a medical device data transfer module operable to receive data from one or more medical devices capable of digitizing medical device output data, and to present said medical device output data to said artificial intelligence module for processing in the diagnosis and treatment of a patient; a medicines supply module operable to automatically query for, receive, and organize inventory information for data collection and analysis in locating and reporting amounts and locations of medicines or medical supplies required by a client in said network; a disaster relief coordination module for receiving disaster-related data from one or more of said network clients at the location of a disaster, and operable to automatically query for, receive, locate, coordinate, and communicate disaster relief personnel or supplies for dispatch to the location of a disaster; a national health delivery module operable as a data presentation module for presenting healthcare recommended courses of action to be implemented across a national healthcare system; a public health key indicator module for addressing identified measures of public health across a national public health system with recommended actions calculated to achieve improvement in said measures with maximal economic effectiveness and shortest time effectiveness; or a global health grant application module operable to coordinate and present healthcare data from any of said modules in a format suitable for review of an economic assistance grant application by a public health economic grant agency or philanthropic organization.

The invention summarized above implements a method for the management and improvement of a national healthcare data management tool, comprising the steps of: receiving and storing medical and healthcare information from a physician or a physician's support staff; identifying a diagnosis and one or more treatment recommendations according to the medical and healthcare information received; storing and organizing the diagnostic and treatment recommendations; retrieving the organized diagnostic and treatment recommendations and processing the diagnostic and treatment for the creation of a national healthcare database, and in which the created national healthcare database is applied to one or more national healthcare purposes selected from the group consisting of: educating medical and healthcare personnel; evaluating the appropriateness of a medical or healthcare worker's individual patient diagnosis and treatment strategy; managing the outbreak of a communicable disease across a national population; referring an individual patient to a physician in a medical specialty; applying digitized data from one or more medical devices to the diagnosis and treatment of a patient; organizing information on the availability of medicines or medical supplies within a national healthcare system; coordinating disaster relief; recommending courses of action to be applied across a national healthcare system for improvement of the system's healthcare delivery; identifying and efficiently addressing factors used to measure the effectiveness of a national healthcare system; or applying for healthcare funding from funding agencies or philanthropic organizations.

In lesser-developed countries around the world, there is a need to achieve improved healthcare outcomes across a given nation's entire national healthcare system. Multiple measurable healthcare outcomes can be improved by a system that can implement multiple system modules in parallel, through the intake of patient and populational medical and health datapoints to create and expand a database, process such datapoints through multiple system modules designed to address different needs or metrics, and integrate processing outcomes into manageable conclusions, summaries, and reports. By relying on a software system, the database becomes the primary tool by which healthcare outcomes are improved, without relying on a particular type, source, or brand of hardware, whether the hardware is being utilized in observation, visualization, diagnosis, processing, recordation, storage, retrieval, transmission, communication, or utilization of healthcare or medical data.

### BRIEF DESCRIPTION OF THE DRAWINGS

FIG. 1 is a simplified block diagram of a general computer system means.

FIG. 2 is a simplified block diagram/flowchart illustrating a general overall processing system.

FIG. 3 is a simplified block diagram/flowchart of a generalized image processing system.

FIG. 4 is a simplified block diagram of an embodiment of a medical software tools platform.

FIG. 5 is a simplified flowchart of an embodiment of an epidemiological information system.

FIG. 6 is a simplified flow chart of applying the system of the invention to the factors comprising a national health system.

FIG. 7 is a simplified flow chart of the organization of system modules to address national healthcare system factors.

FIG. 8 is simplified flow chart of a module of the system for providing medical education.

FIG. 9 is a simplified flow chart of a module of the system for conducting medical provider evaluations.

FIG. 10 is a simplified flow chart of a module of the system for conducting medical provider evaluations of a provider in a remote region lacking communications infrastructure.

FIG. 11 is a simplified flow chart of a module of the system for digital interfacing with medical equipment.

FIG. 12 is a simplified flow chart of a module of the system for providing logistical support to medical providers.

FIG. 13 is a simplified flow chart of a module of the system for its adaptation across multiple languages.

### DETAILED DESCRIPTION OF THE INVENTION

Features of the Invention. The system of the invention has multiple features designed to aid in the delivery of an overall system capable of raising healthcare outcomes across a national healthcare program.

The overall software system architecture is deployable via: remote app, in which a dumb terminal is deployed on the patient-side and connects to a remote processing system, and in which all of the control logics are defined and stored within the remote system; an app that the architecture downloads and directly runs, typically exemplified by mobile systems such as smartphones and tablets and where the architecture provides full control of the processing system, including storage, transmission, and vital sign analysis, and which may also include advanced control logics; a web app, whereby the architecture launches applications, generally written in Java, within a Web browser, though at which point full and/or deep control over the app by the remote user may be lacking; and/or by a web based app, where the architecture interacts with a web-based browser. This adaptability across differing sub-architectures is an important feature of the invention, as in many situations, patient data will be collected remotely at a location that has no internet access, so the data will have to be stored until internet access exists, at which point the data can be communicated for server-mediated processing and subsequent transmission to clients and stakeholders.

Different types of connection are utilized by the system of the invention, including point-to-point connection; multipoint connection; synchronous connections (i.e. e-mail), asynchronous connections (i.e. voice and chat). Information that can be transmitted includes alphanumerical text, voice and sound, e.g. auscultation, static images, and sequences of images and movies.

The system of the invention provides redundant storage, so that failures do not wipe out all the information stored, which is an important consideration under the harsh working conditions typically found in lesser-developed countries, including high temperature, high humidity and precipitation, and high levels of airborne dust. Also, the availability of spare parts is greatly compromised. Storage is available at the server at a greater volume, or at the client, as noted above.

The national healthcare management tool system and method of the invention can utilize open source or proprietary tools, when affordable. At the client level, the use of smart phones and tablets means that the client apparatus is portable and has relatively low power consumption. The system of the invention provides for being able to share and exchange information between function modules, including file formats and remote invocation of procedures and services. The system of the invention is scalable to be easily enriched to cover more cases, wider areas, greater populations, more pathologies, and increasing numbers of health care centers. The system of the invention is structured and documented so as to enable its uptake and use without extensive training or academic education.

In the present invention, since it is a fundamental goal to be able to improve the overall healthcare of an entire country and its national healthcare structure, there are multiple tiers and categories of clients. A non-limiting list of clients can include patients, patient family members, patient friends, physicians, physician assistants, nurses, nurse assistants, nurse practitioners, dentists, audiologists, healthcare aids, orderlies, clinical laboratory technicians, medical equipment technicians, paramedics, search and rescue personnel, pharmacists, pharmacist assistants, clinical administration staff, government administration staff, management, officials, and policy makers, at the local community, regional, national, and international levels, medical students, interns, and residents, and peer-reviewed academic journal editors and reviewers. The system of the invention can additionally be utilized to gather a country's healthcare data, organized by recognized metrics, for inclusion in grant applications to national or international funding agencies and banks, as well as to philanthropic foundations for attracting needed funding for the maintenance, reorganization, expansion of new capabilities, recruitment and retention of medical and healthcare professionals, acquisition of medicines and medical supplies, and for new capital construction.

To deliver on its multiple features, the system of the invention utilizes multiple fields of technology in the invention's architecture and utilization, including, for example, telemedicine, artificial intelligence in medical practice, and video enabled software tools, now discussed in greater detail below.

Telemedicine. Telemedicine is described here as the delivery of health care services, where distance is a critical factor, by all health care professionals using information and communication technologies for the exchange of valid information for diagnosis, treatment and prevention of disease and injuries, research and evaluation, for the continuing education of health care providers, and for the collection, processing and dissemination of health and medical data, all in the interests of advancing the health of individuals and their communities at all geographic levels within a nation.

Whenever distance impairs the proper care to be delivered to a patient, telemedicine can be a good answer. Distance may affect the delivery of the care, both in time and in quality, everywhere. Both developed and developing countries may experience situations where the intervention time, from disease detection to beginning of care, seriously affects the final result of the care itself. Such typical situations range from, for example, acute myocardial infarction to cranial hemorrhage or to wounds or injuries affecting patients in rural locations. Both developed and developing countries may experience situations where quality of care seriously affects the final result of the care itself. As an example, if the resolution of a medical diagnostic image transmitted to the hospital from a rural location is poor, some lesions or anatomical districts affected by the disease or by the injury may go undetected, again compromising the final result of the care. All countries, both developed and developing ones, can benefit from application of telemedicine technology.

According to the definition above, telemedicine can also be concisely referred to as “the use of information and telecommunication technologies (ICT) in medicine” Telemedicine is not only just for remote monitoring or diagnosing a patient, it also includes e-learning techniques (to remotely deliver education both to health care workers and to patients), and teleconsultation (aka telecounseling or teleconsultation, or expert second opinion) services. This latter term refers to any consultation between doctors or between doctors and patients on a network or video link (e.g., Facetime, intranet, Internet, Skype, etc.), as opposed to the “in person” counseling where no ICT is needed to manage the interaction between the patient and the physician(s).

In developed countries several programs have been deployed, for example, in the United Kingdom, Finland, across Europe, and in Taiwan. Some more recent programs were devoted to additionally include medical education, such as in Western Australia. Much less effort has been spent for similar initiatives in developing countries. This is due both to the much smaller return of investment (ROI), to a limited budget available, and to the greater difficulties expected or encountered, also due to the lack of technological infrastructures. Moreover, while telemedicine programs in developed countries in most cases may easily deploy an emergency strategy, such as sending out a helicopter to rescue the patient and to transfer him/her to the nearest hospital in a very short time, similar situations in developing countries are generally more expensive, rarer, and much harder to be deployed. Finally, in developed countries, telemedicine works side-by-side with more conventional health care, completing it, while in developing countries telemedicine in most cases is an alternative, or even the only alternative, to conventional health care. Nevertheless, telemedicine applications in developing countries can provide wide populations with basic health care services and close the distance between rural areas and specialized hospitals usually located in big cities.

Journals on the subject of implementing and operating telemedicine systems include the Institute of Electrical and Electronics Engineers (IEEE) Transactions on Information Technology in Biomedicine; IEEE Journal of Biomedical and Health Informatics (IEEE JBHI); the Journal of Telemedicine and Telecare; the Telemedicine Journal; the International Journal of Medical Informatics; the Applied Clinical Informatics journal, and the Telemedicine and e-Health journal; all of which are known to and readily accessible to those of ordinary skill in the art. The major and most relevant conferences on the same topic consist of, for example, the Annual Conference of the American Telemedicine Association; the European Telemedicine Conference; the Annual International Congress on Telehealth and Telecare; the eTelemed Conference; and the mHealth+Telehealth World Conference. Additionally, useful technical disclosure on telemedine is readily available through commonly available search engines to review on the internet for reports on such projects.

The system of the present invention includes the ability to achieve multiple major goals in the design of a telemedicine system, including the following.

Remote diagnosing and teleconsulting, here meaning that data (including signals and images) are locally (patient-side) acquired and stored, and then forwarded to a main hospital or key clinic, where physicians or their support staff can analyze such data. The remote (physician-side) hospital or clinic will then send back the diagnosis to the patient-side. Support staff on the patient-side or the physician-side can include, for example, nurses, nursing aides, physician assistants, pharmacists, technicians, first aid workers, disaster relief workers, and rescue personnel.

Remote diagnosis can be performed even if the patient is assisted by nurses or other support staff only, and no physician is in the neighborhood: such a situation typically occurs in rural locations of developing countries, and in some cases a preliminary diagnosis is locally performed with the aid of a decision support system (DSS). Teleconsulting, i.e., expert second opinion, is performed among physicians, where a nonspecialist physician requires a remote consultation with one or more specialist physicians; typically, such a situation occurs in emergency centers of rural locations or in minor hospitals of developed countries, or in any location of developing countries.

Remote monitoring system. The patient is monitored in the remote location, his/her signals are continuously acquired, forwarded to the main hospital, and, possibly, locally analyzed by a DSS. If alarms or intervention signals are present, they can be remotely detected and transmitted back to the patient-side. The monitoring system can be managed and locally controlled by a physician or by a nurse.

Remote intervention system. The patient enters the operating room, and the intervention is performed through a local (patient-side) robot that is remotely controlled by a physician in the main hospital. The remote intervention requires that some local assistance be performed by a physician or by a nurse or other support staff.

Remote education (e-learning) system. Students or caregivers (mostly physicians, nurses, and technicians) attend classes taught from remote academic institutions, and possibly by a bi-directional communication interaction with the instructor by asking or sending questions. Remote education can be locally assisted by a local tutor, during and/or after the classes.

Economic investment effort required. An element of standing up a system of the present invention is a consideration and evaluation of the degree of economic investment required and what is available, from government or philanthropic sources. Developed countries feature a much greater gross domestic product (GDP) with respect to the GDP of developing countries. Public expenditure on health, measured as percentage of the GDP devoted to health care, differs even more between developed and developing countries, ranging from a ratio between 5.5 and 7.0 for the richest countries to a ratio between 0.6 and 1.7 for the poorest countries. Some more studies over the decades from 1995 to 2014 report a ratio between 5.9 and 7.7 for high income countries and a ratio between 1.5 and 2.4 for low income countries. It can be easily observed that such a difference in economic resources and investments will characterize any telemedicine project, in terms of complexity, diffusion, accuracy, performance, number of considered cases to mention just few of them, only. Moreover, a low percentage of citizens in developed countries relies on telemedicine as the only medical care system. By contrast, a remarkable percentage of citizens in developing countries look to telemedicine as the only available medical care system, and a measurement of the system's population coverage can be considered a good index of how effective that telemedicine project is.

Location and environment. Two main location types can be classified: rural locations, where needs are mainly devoted to primary health care, including emergency situations and where one will mainly observe first aid centers, often with no specific equipment to manage injured people; and urban locations, where needs are mostly oriented towards chronic pathologies, and where one will expect to observe hospitals with some amount of equipment for signal and image analysis. While citizens in developed countries mainly live in urban locations, a much higher percentage of citizens in developing countries still live in rural locations. A current accredited estimation asserts that some 40% of people in Africa still live in rural locations, and telemedicine is the only chance of obtaining medical care for such persons.

Infrastructure. The main aspects on the infrastructure side of telemedicine cover the pure ICT technological infrastructure, as well as other generic infrastructure elements such as roads, communications signal towers and other equipment, power supply or water supply. A telemedicine system will have to effectively cope with poor-quality electricity supplies, giving as an example that the average electric power consumption in Sub-Saharan Africa is 124 KWh per capita per year, only a tenth of what is to be found elsewhere in the developing world, and barely enough to power one 100 W light bulb per person for three hours a day; poor-quality water supplies; poor-quality telephone services; isolation and lack of continuing medical education (CME) for health care staff; and poor supervision of health care staff. In rural regions of developing countries, communication media mainly include voice (typically, via a VHF transceiver) and text (typically, via e-mail messages), while rural regions of developed countries feature very different and more powerful communication media.

Software requirements. Basic software requirements have to consider both the main location, typically a major hospital running as the server of the telemedicine service, and the remote locations, typically the rural locations and/or the developing countries running as clients of the telemedicine service. Major limiting requirements come from the client side in a server-client architecture. The telemedicine service on the client side can involve one unique remote workstation, e.g., one personal computer (PC), or additional remote workstations, for example a single PC and other biomedical instrumentation, for example a type of bio-signal recorder or a type of an imaging device. Moreover, some local workstations on the client side can be locally connected through a local area network (LAN). The remote server type architecture can be open or proprietary, the operating system can be, typically, Linux or Windows, but the provided services will usually minimally have to provide printer spool, document sharing, proxy, firewall, security and user log-in and authentication services.

Artificial Intelligence in Medicine. The system of the invention utilizes an AI framework means in addressing and delivering multiple outcomes. Such AI frameworks that can be utilized by the system of the invention are commercially available for use or can be created by those of ordinary skill in the art. First, the system of the invention provides a simulation environment for understanding and predicting the consequences of various treatment or policy choices. Such simulation modeling helps improve decision-making and the fundamental understanding of the healthcare system and clinical process—its elements, their interactions, and the end result—by playing out numerous potential scenarios in advance. Secondly, the AI framework utilized provides the basis for clinical artificial intelligence that can deliberate in advance, form contingency plans to cope with uncertainty, and adjust to changing information on the fly. With careful design and problem formulation, such an AI simulation framework approximates optimal decisions even in complex and uncertain environments, and thus outputs conclusions, recommendations, and suggestions for medical intervention that allows a human practitioner to access a storehouse of clinically-based outcomes from which to make the best possible informed decision in concert with the education, experience, and training of the practitioner. Such technology functions in multiple roles: enhanced telemedicine services, automated clinician's assistants, and next-generation clinical decision support systems (CDSS).

In one embodiment, an autonomous AI software means can reside within patient monitoring computation devices and within doctor assisting computation devices. Information from such patient monitoring is communicated to the doctor assisting devices and may influence the doctor through a new recommendation or a change in the treatment decisions or beliefs of the doctor. Such AI software then analyzes the effects of these treatment decisions and delivers updated patient-outcome prediction results to the doctor. In another embodiment, such patient-monitoring and doctor-assisting computation devices function as communication devices to web-based AI software that performs the analysis. Databases of information may be used to help the doctor-assisting computation devices, such as electronic health records, personal history records, genetic marker records, etc.

In other embodiments, an AI means constitutes a method of providing decision support for assisting medical treatment decision-making. For example, a patient agent software module means processes information about a particular patient and a doctor agent software module means then processes information about a health status of a particular patient, informing beliefs relating to patient treatments, and patent treatment decisions. Such AI means also can include the ability to filter information from the patient agent into the doctor agent to create a plurality of decision-outcome nodes, and creating a patient-specific outcome tree with the plurality of decision-outcome nodes. With the patient-specific outcome tree, an optimal treatment may be determined by evaluating the plurality of decision-outcome nodes with a cost per unit change function and outputting the optimal treatment. An exemplary cost per unit change function includes calculating the cost in dollars or local currency that it takes to obtain one unit of outcome change (delta) on a given outcome. The patient agent includes a plurality of health status information at a plurality of times. The doctor agent includes a module that receives rewards/utilities, and a module to select patient treatments in order to maximize overall utilities. The decision-outcome nodes are updated according to a transition model. The method further includes learning wherein when additional information is available, such information is included in a knowledge base used by at least one of either the patient agent software module, or the doctor agent software module, in determining optimal treatment.

Another capability of medical AI is aiding in the implementation that involves a decision support system for assisting medical treatment decision-making. Such a system has a processor and associated memory, with program memory configured to store instructions for enabling the processor to perform operations and storage memory configured to store data upon which the processor performs operations. The storage memory includes data relating to a particular patient. The patient agent software module is for processing information about a particular patient. The doctor agent software module is for processing information about a health status of a particular patient, beliefs relating to at least one of patient treatments and treatment effects, and effects of patient treatments. The software filters information from the patient agent into the doctor agent to create a plurality of decision-outcome nodes and creates a patient-specific outcome tree with the plurality of decision-outcome nodes. An optimal treatment is determined by evaluating the plurality of decision-outcome nodes with a cost per unit change function and outputting the optimal treatment. The optimal treatment is reevaluated when additional information is available from at least one of the patient agent and the doctor agent, after updating the patient-specific outcome tree. The cost per unit change function includes calculating the cost in dollars it takes to obtain one unit of outcome change (delta) on a given outcome. The patient agent includes a plurality of health status information at a plurality of times. The doctor agent includes a module to receive rewards/utilities, and a module to select patient treatments in order to maximize overall utilities. The decision-outcome nodes are updated according to a transition model. A further learning software module has a knowledge base wherein when additional information is available, such information is included in the knowledge base which is used by at least one of the patient agent software module, or the doctor agent software module, and the determining optimal treatment step.

Other exemplary embodiments of medical AI involve a server for providing decision support for medical treatment decision-making. The system comprises a processor and associated memory, the memory including program memory configured to store instructions for enabling the processor to perform operations. The memory also includes storage memory configured to store data upon which the processor performs operations. The storage memory includes data relating to a particular patient, and the program memory includes a plurality of instructions that when executed by the processor enables the processor to execute computer program steps. For example, receiving evidence based information about a health status of a particular patient, doctor beliefs relating to at least one of patient treatments and treatment effects, and patient treatment decisions; filtering the evidence based information to create a plurality of decision-outcome nodes; and determining an optimal treatment by evaluating the plurality of decision-outcome nodes with a scoring function and outputting a message including the optimal treatment.

Such AI medical predictive software systems have a variety of possible uses within the system of the present invention. In one embodiment, such software systems may be centered around a particular patient, so that the physician at the server location, who will be treated here as the attending physician, has additional decision support information with which to formulate a diagnosis and treatment plan to use in comparison with that communicated to the server location by the practitioner at the client location. In another embodiment, such software systems may be centered around a particular type or class of health conditions and/or treatments so that a population of patients may be monitored and general treatment options for the target population can be discerned, which will be particularly useful in the management of communicable diseases, and the reporting of communicable disease outbreaks to public health authorities. In further embodiments, such AI medical software systems may be used with payment or reimbursement systems to allocate resources, e.g. government or insurance payors. Each of these embodiments may be implemented as a server operating by receiving and sending information and messages relating to the aforementioned uses back and forth with any number of clients.

Computational approaches for determining optimal treatment decisions at single timepoints via the use of data mining/machine learning techniques have been the subject of earlier research, for example in C. C. Bennett and T. W. Doub, Data mining and electronic health records: Selecting optimal clinical treatments in practice, Proceedings of the 6th International Conference on Data Mining, (CSREA Press, Las Vegas, Nev., 2010) 313-318; and C. C. Bennett, T. W. Doub, A. D. Bragg, J. Luellen, C. Van Regenmorter, J. Lockman, et al., Data Mining Session-Based Patient Reported Outcomes (PROs) in a Mental Health Setting: Toward Data-Driven Clinical Decision Support and Personalized Treatment, IEEE Health Informatics, Imaging, and Systems Biology Conference, (IEEE, San Jose, Calif., 2011) 229-236, the disclosures of which are explicitly incorporated by reference herein. Initial results of such approaches have achieved success rates of near 80% in predicting optimal treatment for individual patients with complex, chronic illness, and hold promise for further improvement. Predictive algorithms based on such data-driven models are essentially an individualized form of practice-based evidence drawn from the live population. Another term for this is “personalized medicine.”

The ability to adapt specific treatments to fit the characteristics of an individual's disorder transcends the traditional disease model. Prior work in this area has primarily addressed the utility of genetic data to inform individualized care. However, it is likely that the next decade will see the integration of multiple sources of data—genetic, clinical, socio-demographic—to build a more complete profile of the individual, their inherited risks, and the environmental/behavioral factors associated with disorder and the effective treatment thereof. Indeed, a trend is emerging of combining clinical and genetic indicators in the prediction of cancer prognoses as a way of developing cheaper, more effective prognostic tools, for example as disclosed in Sun Y, S. Goodison S, J. Li, L. Liu, and W. Farmerie, Improved breast cancer prognosis through the combination of clinical and genetic markers, Bioinformatics, (2007) 23(1): 30-37; 0. Gevaert, F. De Smet, D. Timmerman, Y. Moreau, and B, De Moor, Predicting the prognosis of breast cancer by integrating clinical and microarray data with Bayesian networks, Bioinformatics, (2006) 22(14):184-190; and A. L. Boulesteix, C. Porzelius, and M. Daumer, Microarray-based classification and clinical predictors: on combined classifiers and additional predictive value, Bioinformatics, (2008) 24(15): 1698-1706, the disclosures of which are incorporated by reference herein.

Such computational approaches can serve as a component of a larger potential framework for real-time data-driven clinical decision support, or “adaptive decision support”, as is shown by role of AI in the system of the present invention. This framework can be integrated into an existing clinical workflow, essentially functioning as a form of artificial intelligence that “lives” within the clinical system, can “learn” over time, and can adapt to the variation seen in the actual real-world population. The approach is two-pronged—both developing new knowledge about effective clinical practices as well as modifying existing knowledge and evidence-based models to fit real-world settings.

Modeling of dynamic sequential decision-making in medicine involves several techniques. Among these modeling techniques are Markov-based approaches, originally described in terms of medical decision-making by J. R. Beck and S. G. Pauker, The Markov process in medical prognosis, Med Decis Making, (1983) 3(4):419-58, the disclosures of which are incorporated by reference herein. Other approaches utilize dynamic influence diagrams or decision trees to model temporal decisions, see Y. Xiang and K. L. Poh, Time-critical dynamic decision modeling in medicine, Comput Biol Med, (2002) 32(2): 85-97; T. Y. Leong, Dynamic decision modeling in medicine: a critique of existing formalisms, Proc Annu Symp Comput Appl Med Care, (AMIA, Washington, D.C., 1993) 478-484; and J. E. Stahl, Modelling methods for pharmacoeconomics and health technology assessment: an overview and guide, Pharmacoeconomics, (2008) 26(2): 131-48, the disclosures of which are incorporated by reference herein. A general overview of simulation modeling techniques is disclosed in Stahl 2008. In all cases, the goal is to determine optimal sequences of decisions out to some horizon. The treatment of time—whether it is continuous or discrete, and (if the latter) how time units are determined—is a critical aspect in any modeling effort, as are the trade-offs between solution quality and solution time. Problems may be either finite-horizon or infinite-horizon. In either case, utilities/rewards of various decisions may be undiscounted or discounted, where discounting increases the importance of short-term utilities/rewards over long-term ones, see A. J. Schaefer, M. D. Bailey, S. M. Shechter, and M. S. Roberts, Modeling Medical Treatment Using Markov Decision Processes, in: M. L. Brandeau, F. Sainfort, and W. P. Pierskalla, eds., Operations Research and Health Care, (Kluwer Academic Publishers, Boston, 2005) 593-612, the disclosures of which are incorporated by reference herein.

Markov decision processes (MDPs) are one efficient technique for determining such optimal sequential decisions (termed a “policy”) in dynamic and uncertain environments, for example in 0. Alagoz, H. Hsu, A. J. Schaefer, and M. S. Roberts, Markov Decision Processes: A Tool for Sequential Decision Making under Uncertainty, Med Decis Making, (2010) 30(4): 474-83, the disclosures of which are incorporated by reference herein, and have been explored in specific medical decision-making problems, for example in S. M. Shechter, M. D. Bailey, A. J. Schaefer, and M. S. Roberts, The Optimal Time to Initiate HIV Therapy Under Ordered Health States, Oper Res, (2008) 56(1): 20-33, the disclosures of which are incorporated by reference herein. MDPs (and their partially observable cousins) directly address many of the challenges faced in clinical decision-making. Clinicians typically determine the course of treatment considering current health status as well as some internal approximation of the outcome of possible future treatment decisions. However, the effect of treatment for a given patient is non-deterministic (i.e. uncertain), and attempts to predict the effects of a series of treatments over time only compound this uncertainty. A Markov approach provides a principled, efficient method to perform probabilistic inference over time given such non-deterministic action effects. Other complexities (and/or sources of uncertainty) include limited resources, unpredictable patient behavior (e.g., lack of medication adherence), and variable treatment response time. These sources of uncertainty may be directly modeled as probabilistic components in a Markov model. Additionally, the use of outcome deltas, averse to clinical outcomes themselves, may provide a convenient history meta-variable for maintaining the central Markov assumption: that the state at time t depends only on the information at time t−1. Currently, most treatment decisions in the medical domain are made via ad-hoc or heuristic approaches, but there is a growing body of evidence that such complex treatment decisions are better handled through modeling rather than intuition alone, for example in P. E. Meehl, Causes and Effects Of My Disturbing Little Book, Journal of Personality Assessment, (1986) 50(3): 370-375, the disclosures of which are incorporated by reference herein.

Partially observable Markov decision processes (POMDPs) extend MDPs by maintaining internal belief states about patient status, treatment effect, etc., similar to the cognitive planning aspects in a human clinician, see V. L. Patel, J. F. Arocha, and D. R. Kaufman, A Primer on Aspects of Cognition for Medical Informatics, J Am Med Inform Assoc, (2001) 8(4): 324-43; and A. S. Elstein and A. Schwarz, Clinical problem solving and diagnostic decision making: selective review of the cognitive literature, BMJ, (2002) 324(7339): 729-32, the disclosures of which are incorporated by reference herein. This helps deal with real-world clinical issues of noisy observations and missing data (e.g. no observation at a given timepoint). By using temporal belief states, POMDPs may account for the probabilistic relationship between observations and underlying health status over time and reason/predict even when observations are missing, while still using existing methods to perform efficient Bayesian inference. MDPs/POMDPs may also be designed as online AI agents—determining an optimal policy at each timepoint (t), taking an action based on that optimal policy, then re-determining the optimal policy at the next timepoint (t+1) based on new information and/or the observed effects of performed actions, for example as disclosed in M. L. Littman, A tutorial on partially observable Markov decision processes, J Math Psycho], (2009) 53(3): 119-25; and S. Russell and P. Norvig, Artificial Intelligence: A Modern Approach, 3rd Ed, (Prentice Hall, Upper Saddle River, N J, 2010), the disclosures of which are incorporated by reference herein.

A challenge in applying MDP/POMDPs is that they involve a data-intensive estimation step to generate reasonable transition models—how belief states evolve over time and observation models—how unobserved variables affect observed quantities. Large state/decision spaces are also computationally expensive to solve particularly in a partially observable setting, and must adhere to specific Markov assumptions that the current timepoint (t) is dependent only on the previous timepoint (t−1). Careful formulation of the problem and state space is necessary to handle such issues.

Embodiments of medical AI known in the art involve MDP/POMDP simulation framework using agents based on clinical EHR data drawn from real patients in a chronic care setting. Optimization of “clinical utility” is done in terms of cost-effectiveness of treatment (utilizing both outcomes and costs) while accurately reflecting realistic clinical decision-making. The focus is on the physician's (or physician agent's) optimization of treatment decisions over time. The results of these computational approaches are compared with existing treatment-as-usual approaches to demonstrate the viability of the AI framework which approaches or even surpasses human decision-making performance.

The framework of embodiments of the invention is structured as a multi-agent system (MAS) for future potential studies, and combining MDPs and MAS opens up many interesting opportunities. For instance, systems may model personalized treatment simply by having each patient agent maintain their own individualized transition model. MAS may capture the sometimes synergistic, sometimes conflicting nature of various components of such systems and exhibit emergent, complex behavior from simple interacting agents, see in another context F. Bousquet and C. Le Page, Multi-agent simulations and ecosystem management: a review, Ecol Modell, (2004) 176(3-4): 31332, the disclosures of which are incorporated by reference herein. For instance, a physician may prescribe a medication, but the patient may not adhere to treatment.

The detailed descriptions of general algorithms and computer means which follow are presented in part in terms of algorithms and symbolic representations of operations on data bits within a computer memory representing biochemical information derived from patient sample data and populated into network models. A computer generally includes a processor for executing instructions and memory for storing instructions and data. When a general purpose computer has a series of machine encoded instructions stored in its memory, the computer operating on such encoded instructions may become a specific type of machine, namely a computer particularly configured to perform the operations embodied by the series of instructions. Some of the instructions may be adapted to produce signals that control operation of other machines and thus may operate through those control signals to transform materials far removed from the computer itself. These descriptions and representations are the means used by those skilled in the data processing arts to most effectively convey the substance of their work to others skilled in the art.

An algorithm is here, and generally, conceived to be a self-consistent sequence of steps leading to a desired result. These steps are those requiring physical manipulations of physical quantities. Usually, though not necessarily, these quantities take the form of electrical or magnetic pulses or signals capable of being stored, transferred, transformed, combined, compared, and otherwise manipulated. It proves convenient at times, principally for reasons of common usage, to refer to these signals as bits, values, symbols, characters, display data, terms, numbers, or the like as a reference to the physical items or manifestations in which such signals are embodied or expressed. It should be borne in mind, however, that all of these and similar terms are to be associated with the appropriate physical quantities and are merely used here as convenient labels applied to these quantities.

Some algorithms may use data structures for both inputting information and producing the desired result. Data structures greatly facilitate data management by data processing systems, and are not accessible except through sophisticated software systems. Data structures are not the information content of a memory, rather they represent specific electronic structural elements which impart or manifest a physical organization on the information stored in memory. More than mere abstraction, the data structures are specific electrical or magnetic structural elements in memory which simultaneously represent complex data accurately, often data modeling physical characteristics of related items, and provide increased efficiency in computer operation.

Further, the manipulations performed are often referred to in terms, such as comparing or adding, commonly associated with mental operations performed by a human operator. No such capability of a human operator is necessary, or desirable in most cases, in any of the operations described herein which form part of the present invention; the operations are machine operations. Useful machines for performing the operations of the present invention include general purpose digital computers or other similar devices. In all cases the distinction between the method operations in operating a computer and the method of computation itself should be recognized. The present invention relates to a system carried out in part through the utilization of methods and apparatus for operating a computer in processing electrical or other (e.g., mechanical, chemical) physical signals to generate other desired physical manifestations or signals. The computer operates on software modules, which are collections of signals stored on a media that represents a series of machine instructions that enable the computer processor to perform the machine instructions that implement the algorithmic steps. Such machine instructions may be the actual computer code the processor interprets to implement the instructions, or alternatively may be a higher level coding of the instructions that is interpreted to obtain the actual computer code. The software module may also include a hardware component, wherein some aspects of the algorithm are performed by the circuitry itself rather as a result of an instruction.

A fuller discussion and illustration of how artificial intelligence can be utilized in the practice of medicine is set forth in the specification and figures of U.S. Pat. No. 10,755,816, the entire teaching and disclosure of which is incorporated herein by reference.

The present invention operates in a computing environment or architecture of the type that have one or more server means and a plurality of client means, exchanging data with the servers. Embodiments of the computing environment may have thousands or millions of client means connected to a network, for example the Internet. Users (not shown) may operate software on one of a client means to both send and receive data and messages over a network via one or more servers and their associated communications equipment and software (not shown), through any number of means well known to those of ordinary skill in the art.

Video Enabled Software Tools. The system software of the present invention also relies on an apparatus for performing these operations. This apparatus may be specifically constructed for the required purposes or it may comprise a general purpose computer means as selectively activated or reconfigured by a computer program stored in the computer means. The algorithms presented herein are not inherently related to any particular computer or other apparatus unless explicitly indicated as requiring particular hardware. In some cases, the computer programs may communicate or relate to other programs or equipment through signals configured to particular protocols which may or may not require specific hardware or programming to interact. In particular, various general purpose machines may be used with programs written in accordance with the teachings herein, or it may prove more convenient to construct more specialized apparatus to perform the required method steps. The required structure for a variety of these machines will appear from the description below.

The present invention may deal with “object-oriented” software, and particularly with an “object-oriented” operating system. The “object-oriented” software is organized into “objects”, each comprising a block of computer instructions describing various procedures (“methods”) to be performed in response to “messages” sent to the object or “events” which occur with the object. Such operations include, for example, the manipulation of variables, the activation of an object by an external event, and the transmission of one or more messages to other objects.

Messages are sent and received between objects having certain functions and knowledge to carry out processes. Messages are generated in response to user instructions, for example, by a user activating an icon with a “mouse” pointer generating an event. Also, messages may be generated by an object in response to the receipt of a message. When one of the objects receives a message, the object carries out an operation (a message procedure) corresponding to the message and, if necessary, returns a result of the operation. Each object has a region where internal states (instance variables) of the object itself are stored and where the other objects are not allowed to access. One feature of the object-oriented system is inheritance. For example, an object for drawing a “circle” on a display may inherit functions and knowledge from another object for drawing a “shape” on a display.

A programmer “programs” in an object-oriented programming language by writing individual blocks of code each of which creates an object by defining its methods. A collection of such objects adapted to communicate with one another by means of messages comprises an object-oriented program. Object-oriented computer programming facilitates the modeling of interactive systems in that each component of the system can be modeled with an object, the behavior of each component being simulated by the methods of its corresponding object, and the interactions between components being simulated by messages transmitted between objects.

An operator may stimulate a collection of interrelated objects comprising an object-oriented program by sending a message to one of the objects. The receipt of the message may cause the object to respond by carrying out predetermined functions which may include sending additional messages to one or more other objects. The other objects may in turn carry out additional functions in response to the messages they receive, including sending still more messages. In this manner, sequences of message and response may continue indefinitely or may come to an end when all messages have been responded to and no new messages are being sent. When modeling systems utilizing an object-oriented language, a programmer need only think in terms of how each component of a modeled system responds to a stimulus and not in terms of the sequence of operations to be performed in response to some stimulus. Such sequence of operations naturally flows out of the interactions between the objects in response to the stimulus and need not be preordained by the programmer.

Although object-oriented programming makes simulation of systems of interrelated components more intuitive, the operation of an object-oriented program is often difficult to understand because the sequence of operations carried out by an object-oriented program is usually not immediately apparent from a software listing as in the case for sequentially organized programs. Nor is it easy to determine how an object-oriented program works through observation of the readily apparent manifestations of its operation. Most of the operations carried out by a computer in response to a program are “invisible” to an observer since only a relatively few steps in a program typically produce an observable computer output.

In the following description, several terms which are used frequently have specialized meanings in the present context. The term “object” relates to a set of computer instructions and associated data which can be activated directly or indirectly by the user. The terms “windowing environment”, “running in windows”, and “object oriented operating system” are used to denote a computer user interface in which information is manipulated and displayed on a video display such as within bounded regions on a raster scanned video display. The terms “network”, “local area network”, “LAN”, “wide area network”, or “WAN” mean two or more computers which are connected in such a manner that messages may be transmitted between the computers. In such computer networks, typically one or more computers operate as a “server”, which is understood to mean one or more computers with large storage devices such as hard disk drives and communication hardware to operate peripheral devices such as printers or modems. Other computers, termed “workstations” or “clients” provide a user interface so that users of computer networks can access the network resources, such as shared data files, common peripheral devices, and inter-workstation communication. Users activate computer programs or network resources to create “processes” which include both the general operation of the computer program along with specific operating characteristics determined by input variables and its environment. Similar to a process is an agent (sometimes called an intelligent agent), which is a process that gathers information or performs some other service without user intervention and on some regular schedule. Typically, an agent, using parameters typically provided by the user, searches locations either on the host machine or at some other point on a network, gathers the information relevant to the purpose of the agent, and presents it to the user on a periodic basis.

The term “desktop” means a specific user interface which presents a menu or display of objects with associated settings for the user associated with the desktop. When the desktop accesses a network resource, which typically requires an application program to execute on the remote server, the desktop calls an Application Program Interface, or “API”, to allow the user to provide commands to the network resource and observe any output. The term “Browser” refers to a program which is not necessarily apparent to the user, but which is responsible for transmitting messages between the desktop and the network server and for displaying and interacting with the network user. Browsers are designed to utilize a communications protocol for transmission of text and graphic information over a worldwide network of computers, namely the “World Wide Web” or simply the “Web”. A non-limiting list of some examples of Browsers compatible with the present invention include the Internet Explorer program sold by Microsoft Corporation (Internet Explorer is a trademark of Microsoft Corporation), the Opera Browser program created by Opera Software ASA, or the Firefox browser program distributed by the Mozilla Foundation (Firefox is a registered trademark of the Mozilla Foundation). Although the following description details such operations in terms of a graphic user interface of a Browser, the present invention may be practiced with text based interfaces, or even with voice or visually activated interfaces, that have many of the functions of a graphic based Browser.

Browsers display information which is formatted in a Standard Generalized Markup Language (“SGML”) or a HyperText Markup Language (“HTML”), both being scripting languages which embed non-visual codes in a text document through the use of special ASCII text codes. Files in these formats may be easily transmitted across computer networks, including global information networks like the Internet, and allow the Browsers to display text, images, and play audio and video recordings. The Web utilizes these data file formats in conjunction with its communication protocol to transmit such information between servers and workstations. Browsers may also be programmed to display information provided in an eXtensible Markup Language (“XML”) file, with XML files being capable of use with several Document Type Definitions (“DTD”) and thus more general in nature than SGML or HTML. The XML file may be analogized to an object, as the data and the stylesheet formatting are separately contained (formatting may be thought of as methods of displaying information, thus an XML file has data and an associated method).

The terms “personal digital assistant” or “PDA”, as defined above, means any handheld, mobile device that combines computing, telephone, fax, e-mail, photographic, and networking features. The terms “wireless wide area network” or “WWAN” mean a wireless network that serves as the medium for the transmission of data between a handheld device and a computer. The term “synchronization” means the exchanging of information between a first device, e.g. a handheld device, and a second device, e.g. a desktop computer, either via wires or wirelessly. Synchronization ensures that the data on both devices are identical (at least at the time of synchronization).

In wireless wide area networks, communication primarily occurs through the transmission of radio signals over analog, digital cellular or personal communications service (“PCS”) networks. Signals may also be transmitted through microwaves and other electromagnetic waves. At the present time, most wireless data communication takes place across cellular systems using second generation technology such as code-division multiple access (“CDMA”), time division multiple access (“TDMA”), the Global System for Mobile Communications (“GSM”), Third Generation (wideband or “3G”), Fourth Generation (broadband or “4G”), personal digital cellular (“PDC”), or through packet-data technology over analog systems such as cellular digital packet data (CDPD”) used on the Advance Mobile Phone Service (“AMPS”)

The terms “wireless application protocol” or “WAP” mean a universal specification to facilitate the delivery and presentation of web-based data on handheld and mobile devices with small user interfaces. “Mobile Software” refers to the software operating system which allows for application programs to be implemented on a mobile device such as a mobile telephone or PDA. A non-limiting partial list of examples of Mobile Software are Java and Java ME (Java and JavaME are trademarks of Sun Microsystems, Inc. of Santa Clara, Calif.), BREW (BREW is a registered trademark of Qualcomm Incorporated of San Diego, Calif.), Windows Mobile (Windows is a registered trademark of Microsoft Corporation of Redmond, Wash.), Palm OS (Palm is a registered trademark of Palm, Inc. of Sunnyvale, Calif.), Symbian OS (Symbian is a registered trademark of Symbian Software Limited Corporation of London, United Kingdom), ANDROID OS (ANDROID is a registered trademark of Google, Inc. of Mountain View, Calif.), and iPhone OS (iPhone is a registered trademark of Apple, Inc. of Cupertino, Calif.), and Windows Phone 7. “Mobile Apps” refers to software programs written for execution with Mobile Software.

“PACS” refers to Picture Archiving and Communication System (PACS) involving medical imaging technology for storage of, and convenient access to, images from multiple source machine types. Electronic images and reports are transmitted digitally via PACS; this eliminates the need to manually file, retrieve, or transport film jackets. The universal format for PACS image storage and transfer is DICOM (Digital Imaging and Communications in Medicine). Non-image data, such as scanned documents, may be incorporated using consumer industry standard formats like PDF (Portable Document Format), once encapsulated in DICOM. A PACS typically consists of four major components: imaging modalities such as X-ray computed tomography (CT) and magnetic resonance imaging (MRI) (although other modalities such as ultrasound (US), positron emission tomography (PET), endoscopy (ES), mammograms (MG), Digital radiography (DR), computed radiography (CR), etc. may be included), a secured network for the transmission of patient information, workstations and mobile devices for interpreting and reviewing images, and archives for the storage and retrieval of images and reports. When used in a more generic sense, PACS may refer to any image storage and retrieval system.

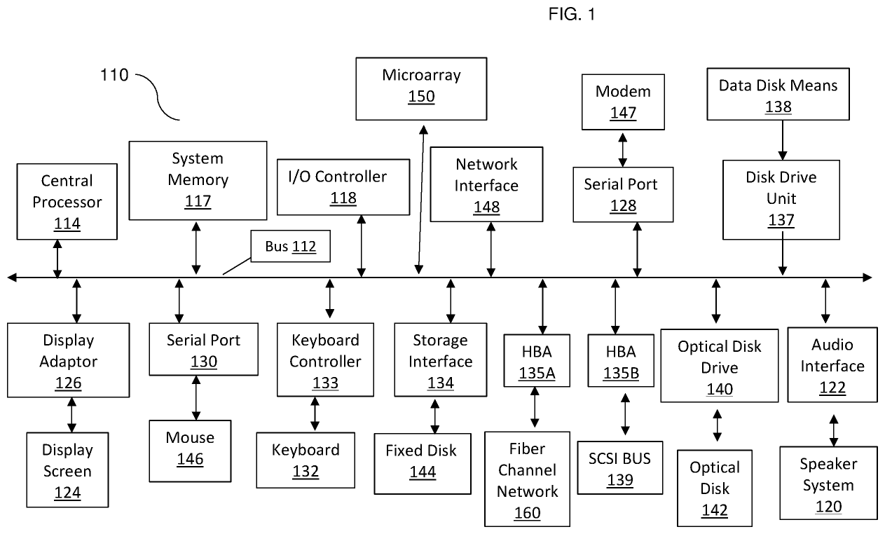

Video Enabled Software Tools. The use of video enabled software tools within the national healthcare management tool system and method of the invention will now turn to the illustration of FIG. 1. FIG. 1 depicts a block diagram of computer system means 110 suitable for implementing a computer server means or computer client means. As this is to be understood as being a computer system means, all hardware features in this FIG. 1 are likewise to be understood as being means for obtaining the function ordinarily associated with such hardware features by those of ordinary skill in the art. Computer system means 110 includes bus 112 which interconnects major subsystems of computer system 110, such as central processor 114, system memory 117 (typically RAM, but which may also include ROM, flash RAM, or the like), an input/output controller 118, an external audio device means, such as for example speaker system 120, enabled via audio output interface 122, an external device, such as display screen 124 enabled via display adapter 126, serial ports 128 and 130, keyboard 132 (interfaced with keyboard controller 133), storage interface 134, disk drive means 137 operative to receive data memory disk means 138, host bus adapter (HBA) interface card 135A operative to connect with Fibre Channel Network 160, host bus adapter (HBA) interface card 135B operative to connect to small computer system interface (SCSI) bus 139, and optical disk drive 140 operative to receive optical disk 142. Also included are mouse 146 (or another point-and-click device, coupled to bus 112 via serial port 130), modem 147 (coupled to bus 112 via serial port 128), and network interface 148 (coupled directly to bus 112).

Bus 112 allows data communication between central processor 114 and system memory 117, which may include read-only memory (ROM) or flash memory (neither shown), and random access memory (RAM) (not shown), as previously noted. RAM is generally the main memory into which operating system and application programs are loaded. ROM or flash memory may contain, among other software code, Basic Input-Output system (BIOS) which controls basic hardware operation such as interacting with peripheral components. Applications resident within computer system 110 are generally stored on and accessed via computer readable media, such as disk drive means (e.g., fixed disk 144), optical drives (e.g., optical drive 140), floppy disk drive means 137, or other storage medium. Additionally, applications may be in the form of electronic signals modulated in accordance with the application and data communication technology when accessed via network modem 147 or interface 148 or other telecommunications equipment (not shown).

Storage interface 134, as with other storage interfaces of computer system 110, may connect to standard computer readable media for storage and/or retrieval of information, such as fixed disk drive 144. Fixed disk drive 144 may be part of computer system 110 or may be separate and accessed through other interface systems. Modem 147 may provide direct connection to remote servers via telephone link or the Internet via an internet service provider (ISP) or satellite uplink/downlink (not shown). Network interface 148 may provide direct connection to remote servers via direct network link to the Internet via a POP (point of presence). Network interface 148 may provide such connection using wireless techniques, including digital cellular telephone connection, Cellular Digital Packet Data (CDPD) connection, digital satellite data connection or the like.

Many other devices or subsystems (not shown) can optionally be connected in a similar manner (e.g., document scanners, digital cameras and so on). Conversely, it is not necessarily the case that all of the devices shown in FIG. 1 be present to practice the present system invention. Devices and subsystems may be interconnected in different ways from that shown in FIG. 1. Operation of a computer system such as that shown in FIG. 1 is readily known to one of ordinary skill in the art and is not discussed in detail in this application. Software source and/or object codes to implement the present disclosure may be stored in computer-readable storage media such as one or more of system memory 117, fixed disk 144, optical disk 142, or floppy disk 138. The operating system provided on computer system 110 may be a variety or version of either MS-DOS® (MS-DOS is a registered trademark of Microsoft Corporation of Redmond, Wash.), WINDOWS® (WINDOWS is a registered trademark of Microsoft Corporation of Redmond, Wash.), OS/2® (OS/2 is a registered trademark of International Business Machines Corporation of Armonk, N.Y.), UNIX® (UNIX is a registered trademark of X/Open Company Limited of Reading, United Kingdom), Linux® (Linux is a registered trademark of Linus Torvalds of Portland, Oreg.), the macOS® Apple® operating system, the iOS® mobile phone and data tablet operating system, or the free and open source software Android operating system for mobile phones and data tablets, or other known or developed operating system. In alternative embodiments, computer system 110 may take the form of a tablet computer, typically in the form of a large display screen operated by touching the screen. In tablet computer alternative embodiments, the operating system may be iOS® (iOS is a registered trademark of Cisco Systems, Inc. of San Jose, Calif., used under license by Apple Corporation of Cupertino, Calif.), Android® (Android as a device is a trademark of Google Inc. of Mountain View, Calif.), Blackberry® Tablet OS (Blackberry is a registered trademark of Research In Motion of Waterloo, Ontario, Canada), webOS (webOS is a trademark of Hewlett-Packard Development Company, L.P. of Texas), and/or other suitable tablet operating systems.

Moreover, regarding the signals described herein, those skilled in the art recognize that a signal may be directly transmitted from a first block to a second block, or a signal may be modified (e.g., amplified, attenuated, delayed, latched, buffered, inverted, filtered, or otherwise modified) between blocks. Although the signals of the above described embodiments are characterized as transmitted from one block to the next, other embodiments of the present disclosure may include modified signals in place of such directly transmitted signals as long as the informational and/or functional aspect of the signal is transmitted between blocks. To some extent, a signal input at a second block may be conceptualized as a second signal derived from a first signal output from a first block due to physical limitations of the circuitry involved (e.g., there will inevitably be some attenuation and delay). Therefore, as used herein, a second signal derived from a first signal includes the first signal or any modifications to the first signal, whether due to circuit limitations or due to passage through other circuit elements which do not change the informational and/or final functional aspect of the first signal.

One peripheral device particularly useful with embodiments of the present invention is microarray 150. Generally, microarray 150 represents one or more devices capable of analyzing and providing clinical, medical, biological, or molecular information from patients. Microarrays may be manufactured in different ways, depending on the number of probes under examination, costs, customization requirements, and the type of analysis contemplated. Such arrays may have as few as 10 probes or as many as over a million micrometre-scale probes, and are generally available from multiple commercial vendors. Each probe in a particular array is responsive to one or more genes, gene-expressions, proteins, enzymes, metabolites and/or other cellular or molecular materials generated by tests conducted in clinical laboratories, and are collectively referred to hereinafter as targets or target products.

It is also possible, in several embodiments, to have multiple types of microarrays, each type having sensitivity to particular expressions and/or other molecular materials, and thus particularized for a predetermined set of targets. This allows for an iterative process of patient sampling, analysis, and further sampling and analysis to refine and personalize diagnoses and treatments for individuals. While a given commercial vendor may have particular platforms and data formats, most if not all may be reduced to standardized formats. Further, sample data may be subject to statistical treatment for analysis and/or accuracy and precision so that individual patient data is a relevant as possible.

The above mentioned and other features and objects of this invention, and the manner of attaining them, will become more apparent and the invention itself will be better understood by reference to the following description of an embodiment of the invention taken in conjunction with the accompanying drawings. Corresponding reference characters indicate corresponding parts throughout the several views. Although the drawings represent embodiments of the present invention, the drawings are not necessarily to scale and certain features may be exaggerated in order to better illustrate and explain the present invention. The flow charts are also representative in nature, and actual embodiments of the invention may include further features or steps not shown in the drawings. The exemplification set out herein illustrates an embodiment of the invention, in one form, and such exemplifications are not to be construed as limiting the scope of the invention in any manner.

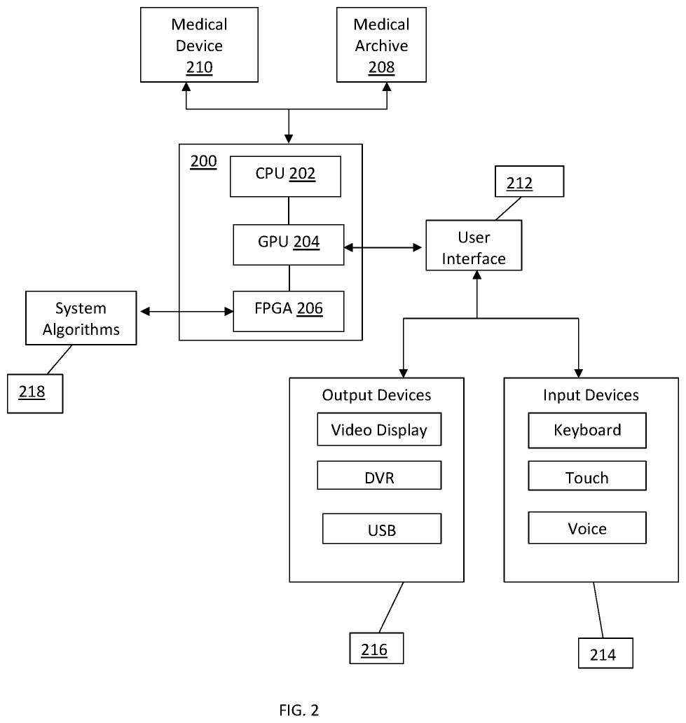

FIG. 2 is a block diagram illustrating an example of the overall processing system that may be used in implementing various features of embodiments of video enabled software tools. In accordance with the preferred embodiment of the present invention, the processing system 200 consists of processor elements such as: a central processing unit (CPU) 202; a graphics processing unit (GPU) 204; and a field programmable gate array (FPGA) 206. The processing system 200 may be used to retrieve and process raw data derived from a medical device 210, which for example can be a surgical camera, or a data storage device, such as a medical archive 208. The surgical camera 210 or medical archive 208 transmits a data stream to the processing system 200, whereby that data is processed by the CPU 202. The FPGA 206, connected to the CPU 202 and the GPU 204, simultaneously processes the received data by using a series of programmed system algorithms 218, thus functioning as an image clarifier within the processing system 200. The GPU 204 communicates with the user interface 212 to display the received data from the medical archive 208. The GPU 204 enables the user interface to then communicate the data to connected input devices 214 and output devices 216. The user interface 212 can communicate to multiple input 214 and output devices 216 simultaneously. An input device 214 can include, for example, a keyboard, touchscreen or voice activated device. An output device 216 can include, for example, a video display, a digital video recorder (DVR) or universal serial bus (USB).

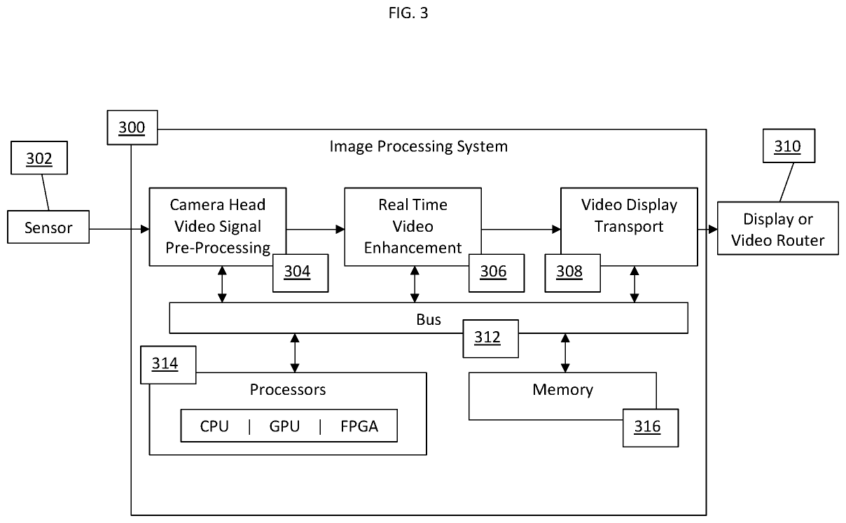

FIG. 3 is a block diagram illustrating a more specific example of one type of video enabled software tool, specifically an image processing system that may be used in implementing various features of embodiments of the disclosed technology. In accordance with the preferred embodiment of the present invention, the image processing system shown generally at 300 consists of three components that process image data received from a sensor 302 in order to send that data to a display or video router 310. The three components of the image processing system 300 are: camera head video pre-processing 304; real time video enhancement 306; and the video display transport 308 function. Image data is collected by a sensor imaging device 302, and is then transmitted to the camera head video pre-processing component 304 within the image processing system 300. This data may be, for example, a raw video image that is pre-processed using various image processing algorithms. Image pre-processing may also include software modules for image registration and segmentation to optimize the video data and communicate via the system bus 312 with the internal system processors 314: the CPU; GPU; and FPGA. The pre-processed image data is transmitted to the real-time video enhancement 306 component, whereby the image data is enhanced to improve clarity or highlight certain details. Once the image data resolution has been enhanced, the video display transport 308 component completes image post-processing, formatting from the initial sensor resolution to the eventual display resolution, for example, enhancing the video data to 1080p HD or 4K display resolution or using software modules such as video cross conversion, scaling and adding graphic overlays. The processed image data is then transmitted from the image processing system 300 to the display or video router 310. The video display transport also saves the processed image data to the processing system memory 316 that can consist of internal and external memory storage.

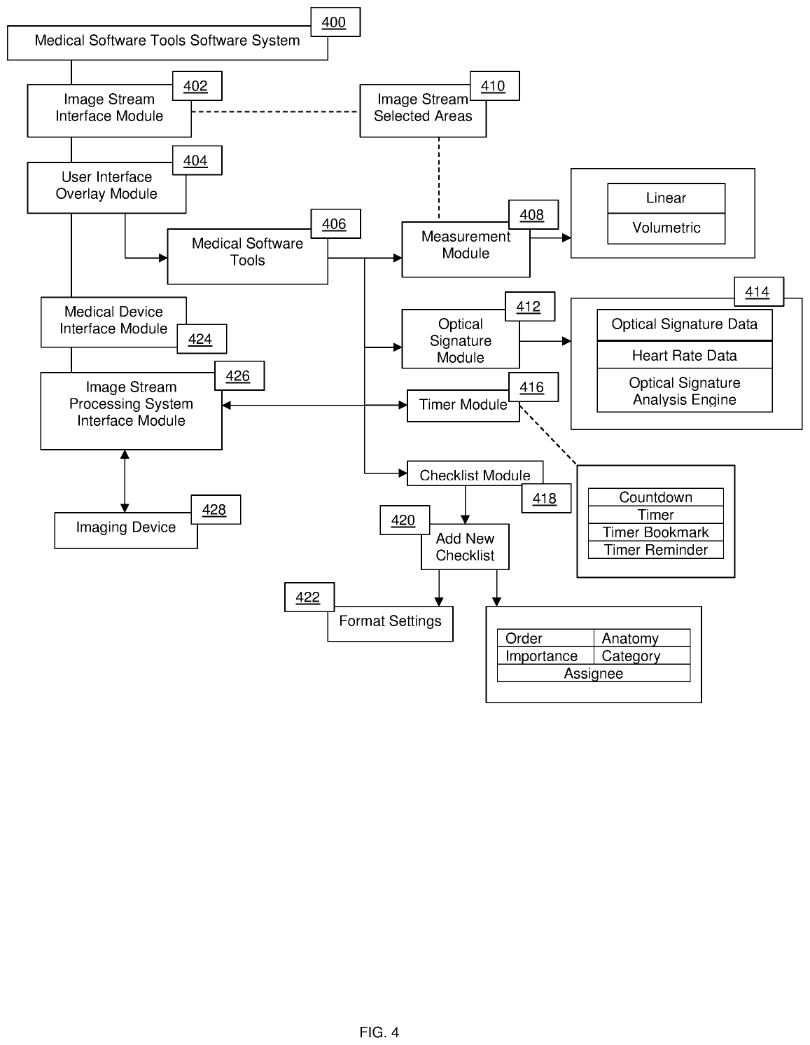

FIG. 4 is a block diagram, more specifically, of a video enabled tool technology, namely one for illustrating the measurement, optical signature module, timer and checklist modules within the medical software tools platform. In accordance with the preferred embodiment of the present invention, the medical software tools platform system 400 may receive an image stream from an image stream processing system through the image stream interface module 402. A specific area of the overall image stream can be selected 410 and utilized within the user interface overlay module 404, which may include one or more graphical user interface (GUI) elements presented over the image stream received through the image stream interface module 402. The user interface overlay module 404 enables communication between the medical software tools platform system 400, and one or more of the medical software tools 406, such as the measurement module 408, the optical signature module 412, timer module 416, and checklist module 418.

The medical device interface module 424 may facilitate communication between the medical software tools platform system 400, and one or more of the medical software tools 406, such as the measurement module 408, optical signature module 412, timer module 416, and checklist module 418. The measurement module 408 may facilitate measurement of one or more anatomical structures or tissue presented in the content of an image stream received through the image stream interface module 402. Depending on the embodiment, the measurement module 408 may enable a user (e.g., surgeon) to select a region in the image stream 410 and determine a measurement based on the selected region. The measurement may include linear measurements (e.g., width, height, length) and volumetric measurements of an anatomical structure or tissue delineated by the selected region.

The optical signature module 412 may facilitate the processing of signature data 414 such as optical sensor data, heart rate data and the optical signature analysis engine. The timer module 416 may facilitate the addition of one or more countdown timers, clocks, stop-watches, alarms, or the like, that can be added and displayed over the image stream through the user interface provided by the user interface overlay module 404. For example, the timer module may allow a user (e.g., surgeon) to add a countdown timer in association with a surgical step (e.g., clamping an artery). For example, a countdown timer may be associated with a specific blood vessel that must be temporarily clamped during surgery but must be opened within a small window of time. A user may be able to select from a list of pre-defined countdown timers, which may have been pre-defined by the user. A clock when added may be used as a time bookmark during surgical procedures. The timer module 416 may communicate with an image stream processing system interface module 426 utilized in an operating room to process an image stream acquired by an imaging device 428.

The checklist module 418 may enable a user (e.g., surgeon) to add and maintain a checklist in connection with a medical procedure 420. For example, the checklist module 418 may provide a list of checklist items for a medical procedure. Each checklist item may indicate whether a step of the medical procedure has been completed or has yet to be completed. The checklist module 418 may allow a user to present the checklist in different ways using the checklist module formatting settings 422. For instance, the checklist items may be organized and presented according to their procedural order, their importance, their relation to a patient's anatomy, their category, or their assigned individual (e.g., whether a given checklist item is the nurse's responsibility versus the surgeon's responsibility). In another example, the checklist items may be presented by using different visual structures, such as a tree structure or a scrolling list.

The operation of individual modules of the national healthcare management tool system and method in the support of national healthcare assessment factors are now illustrated in the following examples with the aid of several of the accompanying Figures.

### Example 1

Delivery of Epidemic and Pandemic Medical Information

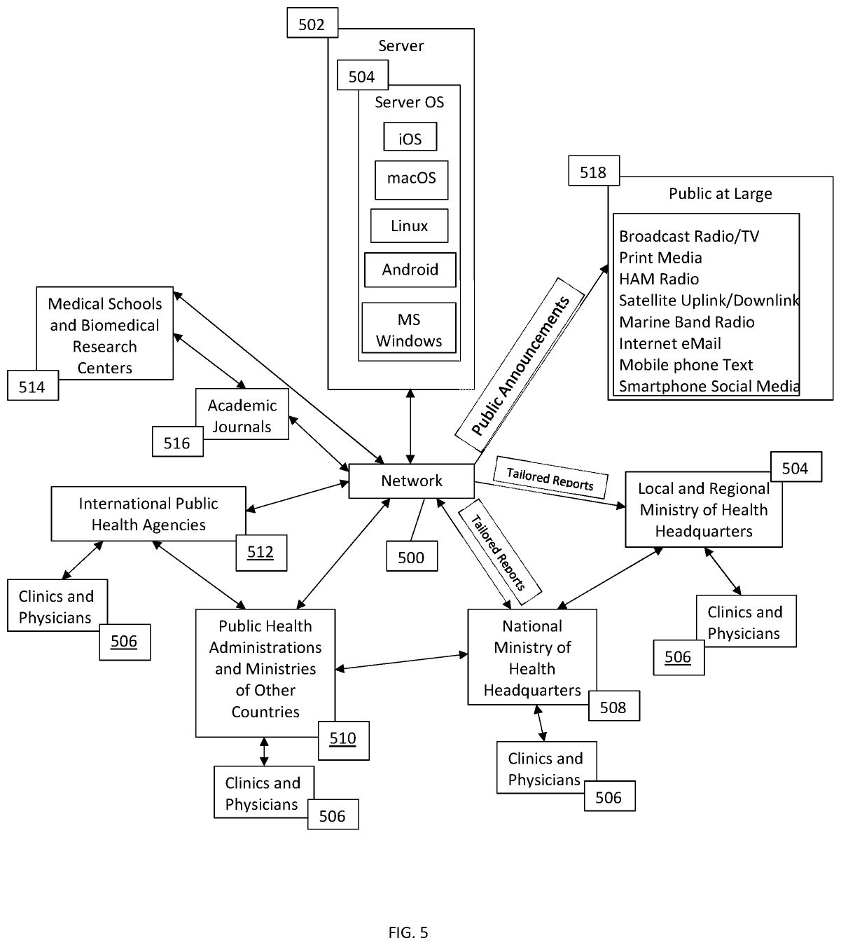

FIG. 5 is a block diagram illustrating how the national healthcare management tool system and method of the invention delivers health and medical data to all stakeholders throughout a country's national health system, and beyond. The network, 500, is enabled by a server means 502, which represents one or more server computing means, including some suitably sized server facility or farm operating any number of individual servers. Server means 502 has been designed to run any common operating system software of choice on server operating system platform 504. A non-limiting list of examples of such operating systems include a variety or version of either MS-DOS® (MS-DOS is a registered trademark of Microsoft Corporation of Redmond, Wash.), WINDOWS® (WINDOWS or MS-WINDOWS is a registered trademark of Microsoft Corporation of Redmond, Wash.), OS/2® (OS/2 is a registered trademark of International Business Machines Corporation of Armonk, N.Y.), UNIX® (UNIX is a registered trademark of X/Open Company Limited of Reading, United Kingdom), Linux® (Linux is a registered trademark of Linus Torvalds of Portland, Oreg.), the macOS® Apple® operating system, the iOS® mobile phone and data tablet operating system, or the free and open source software Android operating system for mobile phones and data tablets, or other known or developed operating system.

Network 500, enabled by server means 502, is structured in this illustration so as to be a hub to multiple client stakeholders, and will send and receive health, medical, and epidemiological data. The FIG. 5 network and its clients illustrate the transmission of data on communicable diseases, natural disasters, and relatively large-scale datasets useful in the assessment of a country's healthcare system and its response to policy decisions or investments of financial resources. Thus for example, data generated by an outbreak of dengue fever will be processed and forwarded to local and regional ministry of health offices or headquarters 504, who in turn will communicate such information to and from physicians and clinics within its region 506. Communications to such headquarters 504 can be advantageously tailored to the known local resources and needs of the regional clinics 506. As information on how a dengue fever outbreak is gathered at local clinics 506, it is sent back to local headquarters 504 and in turn sent back to the network server 500 for storage, evaluation, processing, and for being re-transmitted in raw, evaluated, or assessed form; in turn, from server 500, the information is transmitted to a national ministry of health headquarters 508 that can maintain two-way communication with server 500, or can transfer information to the nation's clinic's and physicians 506, or can transmit to the national public health administrations and ministries of other countries 510.

International public health agencies 512 can receive this information from the network 500, or from the public health administration and ministries of countries 510, and can issue the appropriate bulletins, reports, cautions, or warnings globally, as well as sending the information to clinics and physicians 506 directly who have set up subscriptions, watches, or notification means with the international agencies 512.

The network 500 can also communicate such information to medical schools and biomedical research centers 514, either directly, or through academic journals 516. Although not illustrated, insurors and other third party payors can also be connected with the network, as they have a commercial interest in being kept appraised of developments that can impact their insureds.

Of particular interest with regard to information about not only communicable disease, but also about the effects of natural disasters and rescue and relief efforts, the network 500 can generate public announcements and bulletins to the general population of a country 518, through a number of communications means. Communications means for such announcements include, for example, broadcast radio and television, print media, Internet emails, smartphone and tablet social media, print media, satellite uplink and downlink, marine band radio, and HAM shortwave radio.

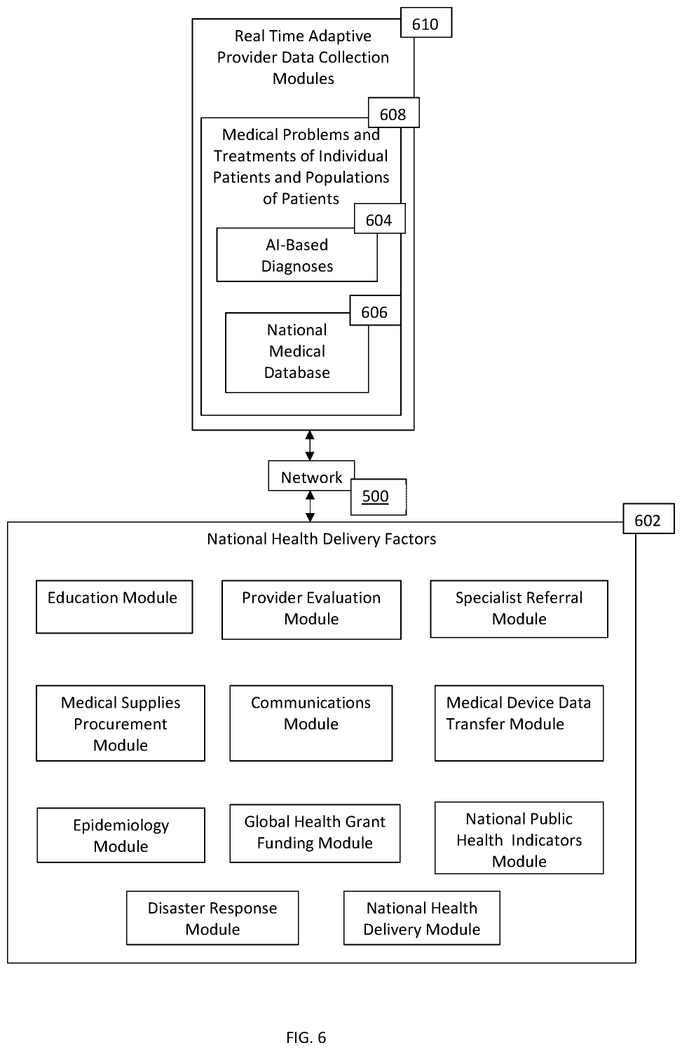

The ability of the software system of the invention that has been disclosed in FIG. 5 above, to effectuate the communication of information on epidemiological events, is just one of at least twelve different factors in evaluating and improving the state of healthcare across a national healthcare system. The factors we have identified are: medical and health education; the evaluation of health and medical provider's delivery of services; the ability to arrange for referrals of patients to medical specialists; the procurement of medical supplies and medicines; the ability to communicate data generated by medical devices; the aforesaid epidemiology information management; responses to natural disasters; the ability to deliver health across a national system; the ability to measure and then improve on national public health indicators; the ability to apply for and obtain funding from government agencies and philanthropic organizations; and the ability to coordinate communications across all of these factors. These factors are illustrated in FIG. 6. The network 500 implements the delivery of the national health factors, which collectively are illustrated at 602, and the delivery is enabled by the ability to generate AI-based medical diagnoses 604 and a steadily expanding database of the nation's health data in a national medical database 606, which in turn are the means by which the country's aggregate medical problems and the collected data on both individual patient treatments and on collective populations of patients 606 are generated and applied to the creation, maintenance, and growth of an adaptive system of real time provider data collection modules 610 that are configured to deliver on the above-identified national health factors in evaluating and improving a country's healthcare system.

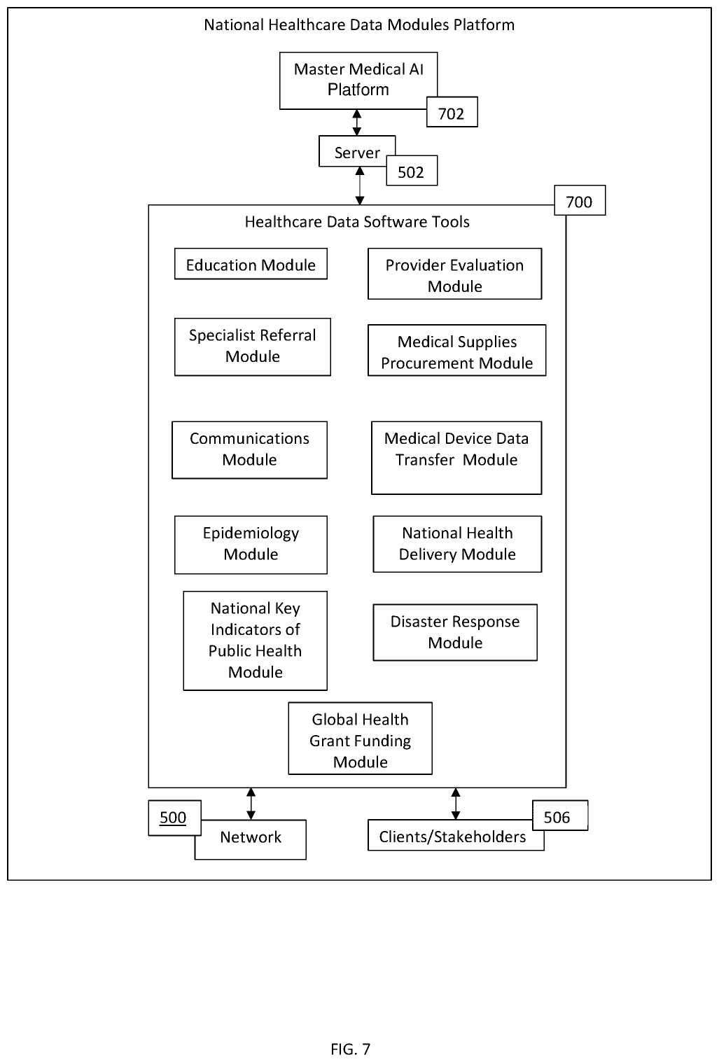

The identified factors in assessing and improving the state of healthcare in a country need to be supported by customized software modules that can both directly address the need to manage the factor, and that have to be able to communicate with all of the other modules in the system, thereby addressing all of the factors in parallel and in real time. Thus, FIG. 7 illustrates the manner in which the identified national health factors 602 are digitally processed by a parallel number of software modules 700 that collectively form a data processing platform. Network server means 502 processes data from the individual modules using the master medical AI platform 702, which is the system software that is the data processing counterpart of the real time adaptive provider data collection modules 610. Master AI platform 702, via the server means 502, effectuates the multiple software modules that correspond to the health delivery factors identified above. Thus, each factor identified in FIG. 6 has a counterpart software module in FIG. 7. The modules collectively make up healthcare data software toolset 700 that in turn generates the data outputs communicated to network 500 and either directly or indirectly to physicians and clinics 506, and other stakeholders.

### Example 2

Delivery of Medical and Health Education

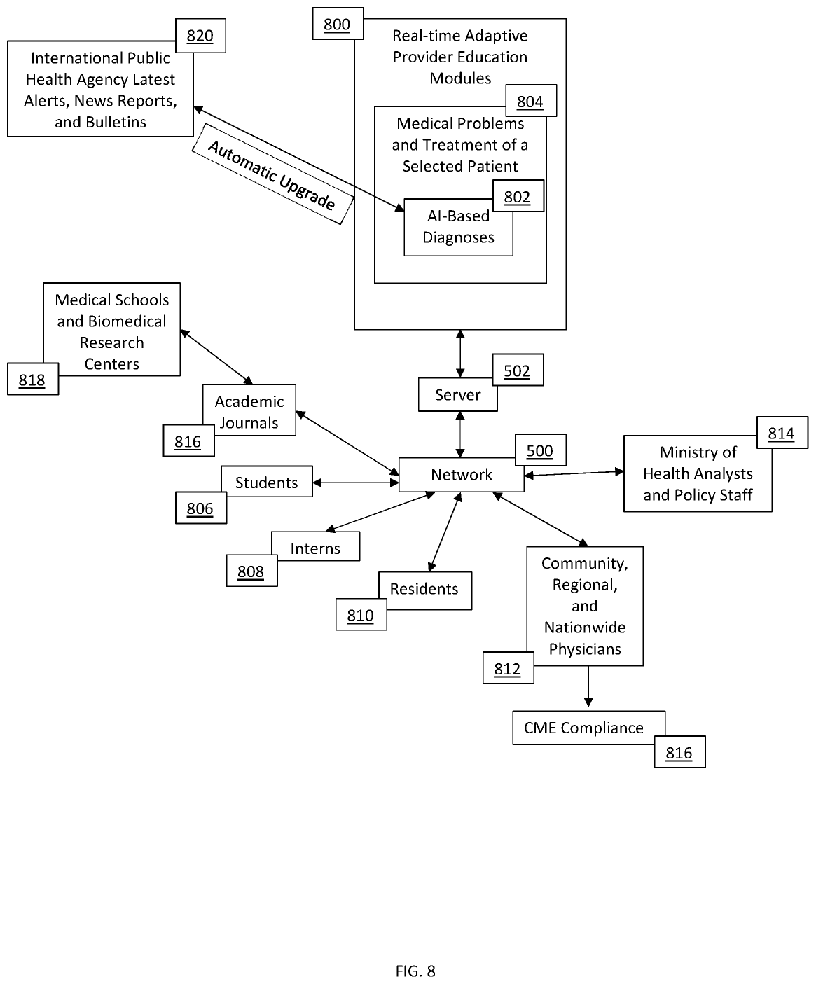

FIG. 8 discloses a preferred embodiment of the present invention, in which the national healthcare factor of adequate medical and healthcare education is enabled through the operation of the real-time adaptive provider education module or modules 800. Education module 800 operates not only to disseminate principles of the practice of medicine and best practices in health, it utilizes an AI-Based Diagnosis software app 802 that receives clinical information about either an individual patient or a group or population of patients, analyzes the clinical data, derives conclusions about the diagnosis of the patient or patients and generates one or more therapeutic treatment recommendations 804, communicates them through server 502 over network 500 to a number of clients and stakeholders. Such clients and stakeholders can include medical students 806, medical interns 808, medical residents 810, community, regional, or nationwide physician groups or networks 812, ministry of health analysts and policy-making staff 814, academic medical journals 816, and medical schools and biomedical research institutions 818. All of these clients are groups that in the practice of medicine and in the delivery of healthcare benefit from learning through observation of case studies—the study of how a clinical presentation leads to a diagnosis, and how one or more therapeutic interventions performs in terms of a beneficial or positive outcome for the patient. In a developing country, such stakeholders are generally widely distributed by significant distances over terrain that makes travel difficult. Their being in such remote locations makes an electronic system of educational materials necessary, but in the practice of the software system of the present invention, they receive not only didactic materials, but the benefit of AI-generated “second opinions” on diagnosis and treatment they that can then apply to future patient presentations in their respective clinical situations. All of the communications links are bi-directional, signifying that clinical data travels back and forth in a manner in which both the AI software and the persons in communication with it are learning the clinical situations, and learning from one another.

The AI-Based Diagnosis software app module 802 is additionally linked to international public health agency-released reports, alerts, and bulletins 820, which functions to feed the most up-to-date data on medical and health news into the module 802, thereby expanding its database and its decision-making scope for the benefit of all clients and stakeholders.

Of great importance in a national healthcare system is the ability of licensed practitioners to obtain certification of ongoing continuing medical education (CME). Educational matter generated by module 802 that is received by physicians 812 can be utilized to obtain such CME and even demonstrate compliance with CME requirements 816.

### Example 3

Delivery of Provider Evaluations

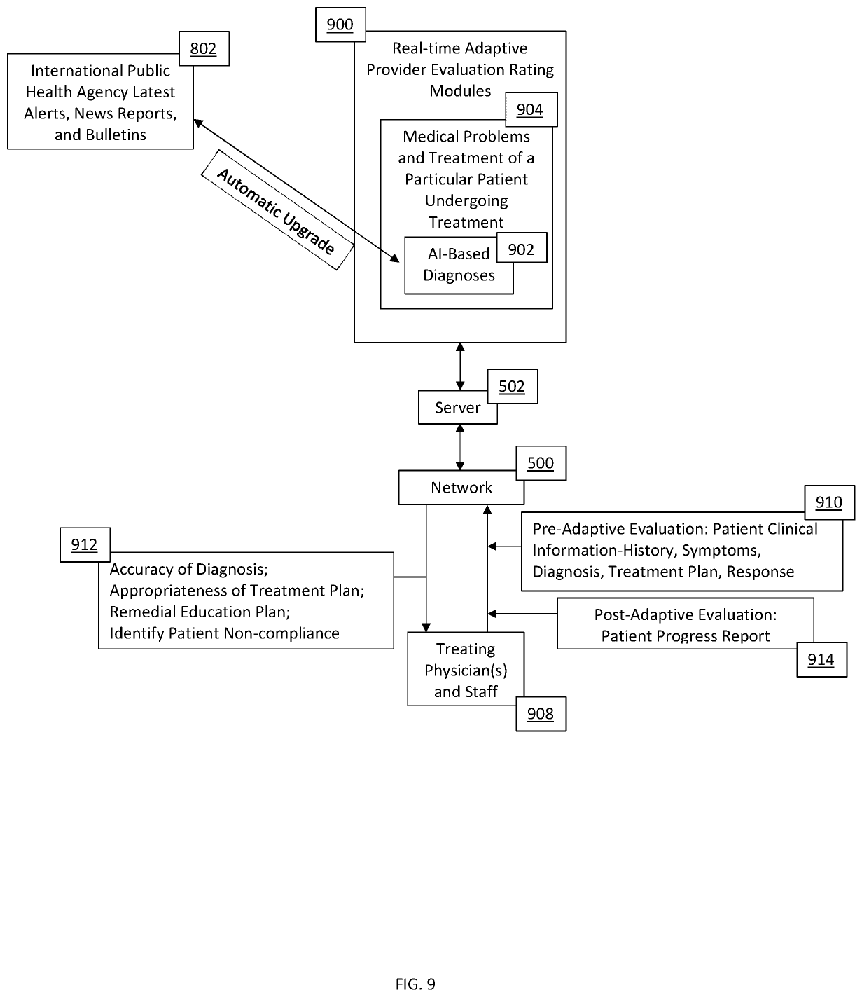

Closely related to the delivery of medical and health education is the delivery of provider evaluations. The correctness of a physician's diagnosis and the effectiveness of that physician's choice of therapeutic intervention benefits the patient and the physician when those diagnostic and therapeutic decisions can be effectively reviewed, evaluated, possibly re-directed, and communicated back to the practitioner in the field. When the aggregate of such reviews and re-directions as well as the clinical results are collected, stored, and called upon by an AI-assisted national database that can perform all of these activities electronically in real-time, then a key factor in the assessment of the quality of a national healthcare system is thereby supported and enhanced. In FIG. 9, a software module similar to 800 is shown by element 900, which is a real-time adaptive provider evaluation rating module or modules. Evaluation module 900 operates not only to review and assess physician or other practitioner diagnostic and therapeutic conclusions, it improves progress toward best practices in medicine and health, by utilizing an AI-Based Provider Evaluation software app 902 that receives clinical information about an individual patient, analyzes the clinical data, derives conclusions about the appropriateness of the diagnosis of the patient and generates one or more therapeutic treatment recommendations 904, and communicates them through server 502 over network 500 to the treating physician or physicians and their staff 908. Treating physician 908 initiates the process by communicating the patient's pre-adaptive evaluation 910 that includes in the clinical presentation the patient's symptoms, basics such as blood pressure, weight, heart rate and other basic clinical information, as well as particularized clinical information such as output of a medical device or an image, clinical laboratory blood or urine specimen data or when available, the patient's medical history, the physician's initial diagnosis and treatment plan, and to the extent it is known, the patient's early response to the plan. All such patient inputs 910 are digitized and delivered via network 500 and its server means 502 to the provider evaluation app 900. The provider evaluation app 900 will process the received patient data and then communicate to physician 908 the app's assessment 912 of the accuracy of the original diagnosis; the appropriateness of the treatment plan; the possible need for additional remedial continuing medical education; and its assessment of the degree of the patient's compliance with the original therapeutic intervention (patient non-compliance is the single greatest problem in therapeutics). Following receipt of the app's assessment 912, the plan is applied to the patient, and in a post-adaptive evaluation, the patient's progress is reported 914 back to the evaluation app 900, where it becomes part of an ever-expanding national medical and healthcare database for the evaluation app 900 to draw upon in all future calculations. This ever-expanding database of national medical and healthcare, however, is additionally drawn upon by all other modules in the software system of the invention to achieve progress toward the aforementioned factors in the assessment and improvement of the country's national healthcare system.

### Example 4

Delivery of Provider Evaluations to Remote Places

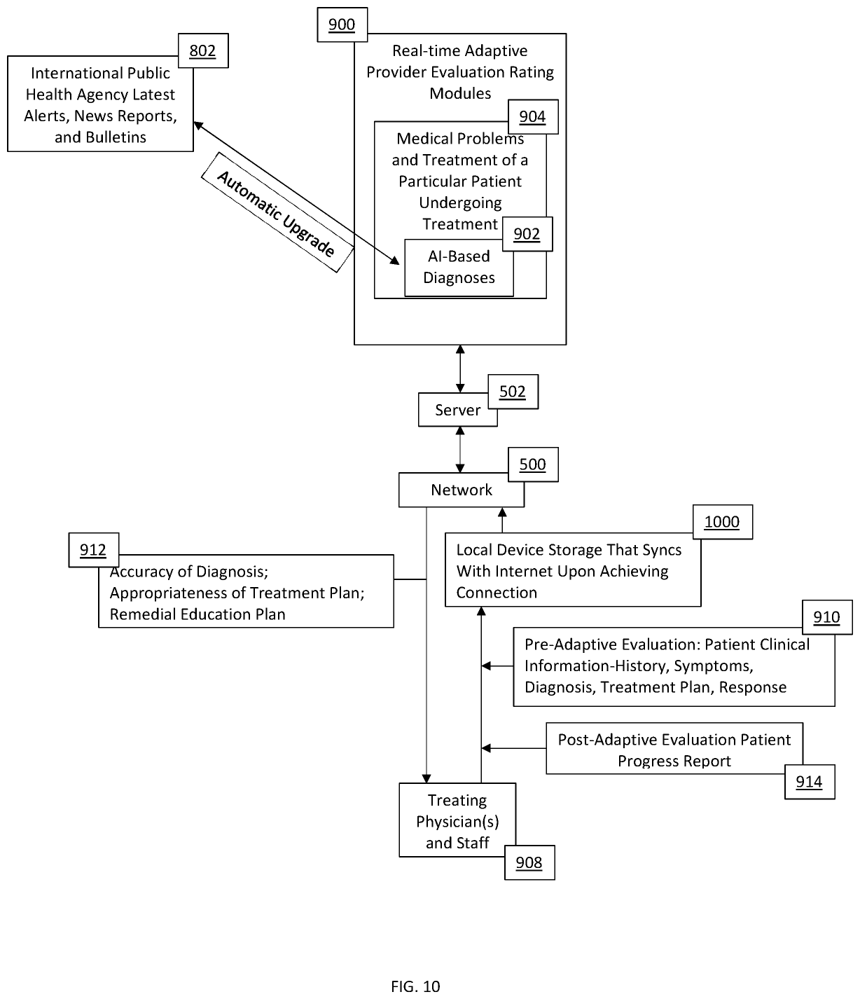

However, many if not most of the developing countries of the world are characterized by limited wireless communications access to the internet or to a landline data communications means. As it is very likely that a treating physician or their staff will be performing their duties in an area not accessible to, or by, the internet or landline means, an additional feature of the provider evaluation app 900 is that the collected clinical data on a patient and the physician's pre-adaptive evaluation 910 can be stored in whatever data communication device is at hand, for example a smart phone, a tablet, a laptop, or a transportable personal computer symbolized by FIG. 10, element 1000, and then be available for downloading and communicating to the network 500 when wireless or landline internet access does become available, and then receiving the module 900's assessment in, or close to, real time, depending on processing speed and capacity.

### Example 5

Delivery of Interfaced Communications With Medical Devices and Cameras

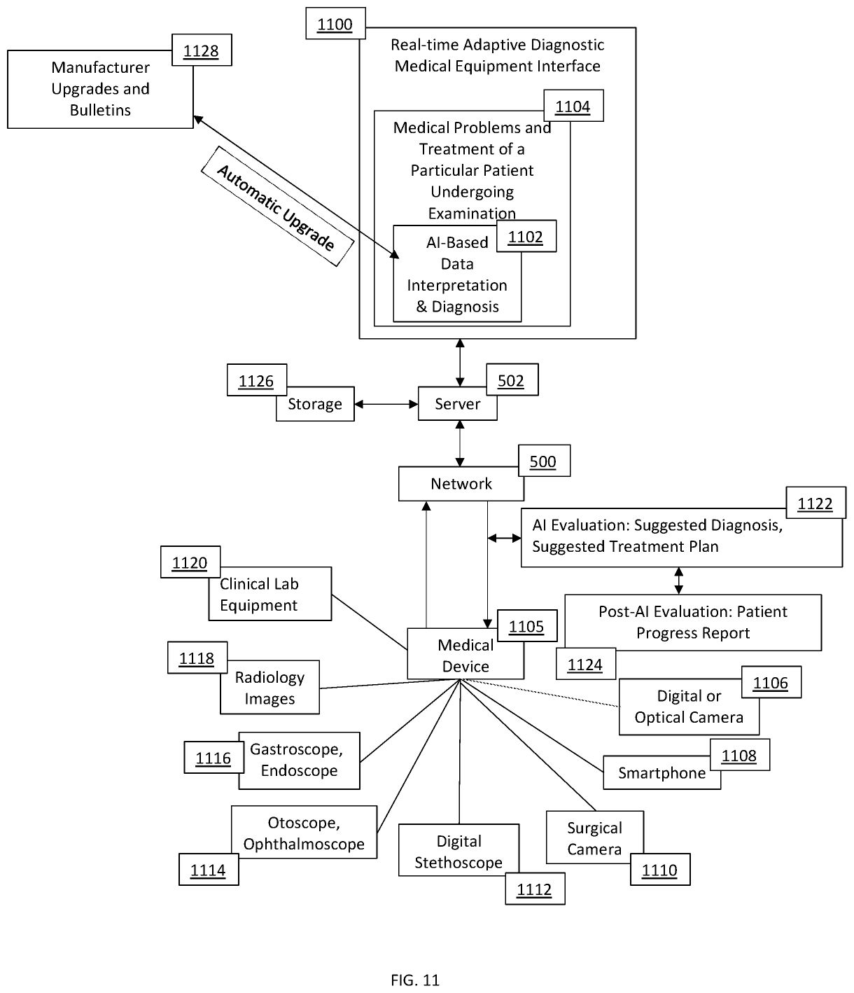

Another factor in the assessment and improvement of healthcare across a national health system is the ability to digitize and communicate the outputs of medical devices and cameras that are being used in the diagnosis and assessment of a patient. FIG. 11 discloses a preferred embodiment of the software system of the present invention in which a module of the system enables the collection, transmission, observation, assessment, and storage of digital data collected from a medical device or camera. Such devices are present throughout all areas of the practice of medicine and it is an important component of an integrated national health system that they can effectively communicate with not only the users that are present by reading their screens or monitors, but that they can also be electronically connected with remote locations for real-time digital processing of their outputs in the assessment of a patient's symptoms, diagnosis, and therapy. At FIG. 11 there is disclosed a real-time adaptive diagnostic medical equipment interface module 1100, which is another counterpart module to modules 800 and 900 described above. Module 1100 operates not only to intake and evaluate digitized signals from medical equipment, it utilizes AI-based data interpretation and diagnosis module 1102, which receives digitized output signals about a patient or a group or population of patients, analyzes the device output data, derives conclusions about the diagnosis of the patient or patients and assists the AI-based diagnosis module 900 in generating therapeutic treatment recommendations, and communicates them through server 502 over network 500 to the treating physicians or their staff. A treating physician or their staff initiates the process by using a medical device 1105 in their examination of their patient. A non-limiting list of exemplary medical devices is illustrated so as to show a digital or optical camera 1106, a smartphone equipped with a digital camera 1108, a specialized surgical camera 1110, a digital stethoscope 1112, a digital otoscope or ophthalmoscope 1114, a gastroscope or endoscope 1116, an x-ray machine or other device generating radiological images 1118, or clinical laboratory equipment means 1120. Any of these devices that have been manufactured with the ability to digitize and communicate output patient data, which then transmitted to the network 500 and within server 502 is processed by the medical equipment interface module 1100. The system then returns the AI-derived interpretation and diagnosis based on the submitted digital medical device back to the treating physician and their staff with a suggested diagnosis 1122 and suggested treatment plan, and is written so as to receive patient progress reports 1124, which in turn can generate new diagnoses or new treatment interventions as may be called for. The data is archived in storage means 1126, and is then fed into the national health system database for reference in the management of future cases and for developing country-wide models of various aspects of the health of the population. Finally, the medical equipment interface module 1100 is configured to be able to receive product updates and bulletins from the manufacturers 1128 of the various medical devices.

### Example 6

Specialist Referrals, and Medicines and Supplies Information

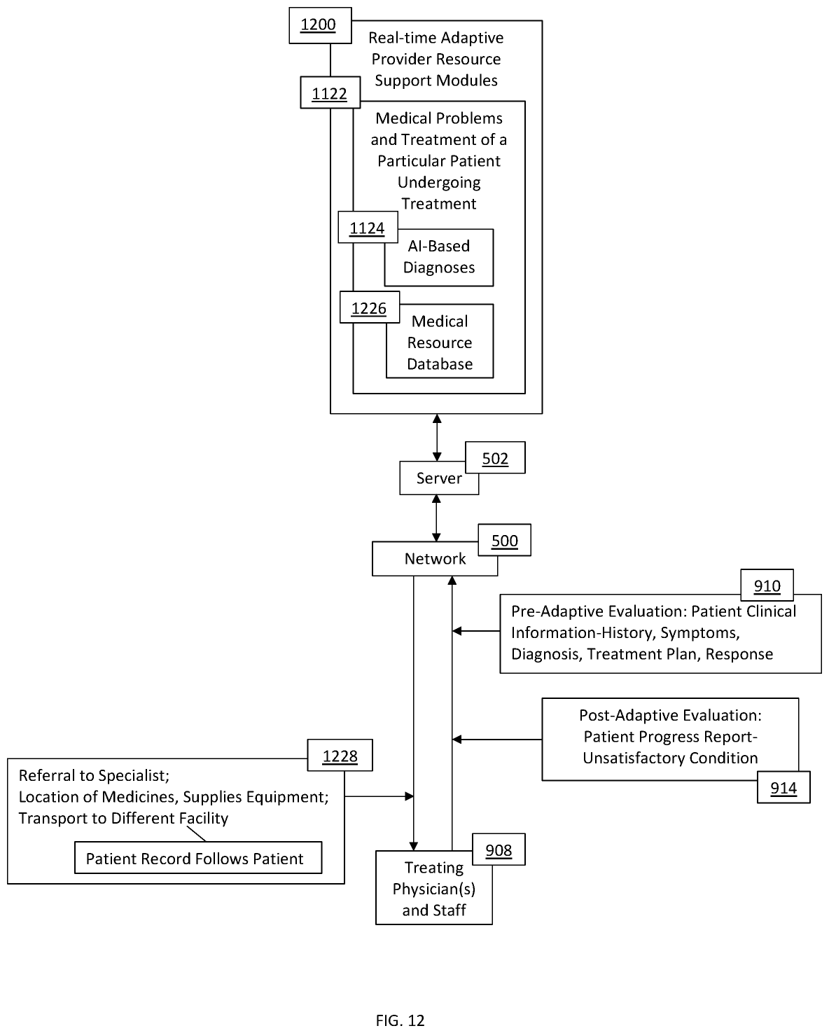

National healthcare systems are also measured by their ability to get patients to a specialist physician, to locate supplies of pharmaceutical medicines, and to locate inventories of needed medical supplies. FIG. 12 discloses how a software module of the system of the invention, written to provide real-time locations of specialists, medicines, and medical supplies, interacts with physicians and their staff in the field. In FIG. 12, a software module similar to 800 is shown by element 1200, which is a real-time adaptive provider resource support module or modules. Treating physician 908 initiates the process by communicating the patient's pre-adaptive evaluation 910 that includes in the clinical presentation the patient's symptoms, basics such as blood pressure, weight, heart rate and other basic clinical information, as well as particularized clinical information such as output of a medical device or an image, clinical laboratory blood or urine specimen data or when available, the patient's medical history, the physician's initial diagnosis and treatment plan, and to the extent it is known, the patient's response to the plan. All such patient inputs 910 are digitized and delivered via network 500 and its server means 502 to the resource support module 1200. Resource support module 1200 operates so as to help treating physicians and their staff in the field to locate medical specialist physicians, arrange for examinations, arrange for patient transportation, locate sources of a medication needed to treat the patient, and to locate sources of medical supplies needed by the patient or by the local clinic itself. Module 1200 carries out these functions by utilizing an AI-Based Diagnosis software app 1224 that receives clinical information 910 about an individual patient, analyzes the clinical data, derives conclusions about the appropriateness of a referral to a specialty physician, queries the medical resource database 1226, then checks on a specialist's location and schedule and generates and communicates a referral recommendation 1228, or the location of medicines, or the location of medical supplies, or the availability of transportation means for the patient's transit to a different facility, communicates them through server 502 over network 500 to the treating physician or physicians and their staff 908. If the patient does transit to a different facility, that patient's medical record either travels with the patient, stored on a laptop computer, tablet, or smartphone to be downloaded at the receiving facility, or is uploaded to a secure file in the national patient database 606 and then downloaded from the medical database to the receiving facility. The system reports follow-up information on the patient for evaluation by the module 1200 as to whether a return visit or consultation with the specialist is warranted.

In a similar flow of data, resource support module 1200 can query the medical resource database 1226 for the location of medicines or medical supplies that are needed by the patient or by the clinic.

### Example 7

Acceptance Uptake in Different Countries

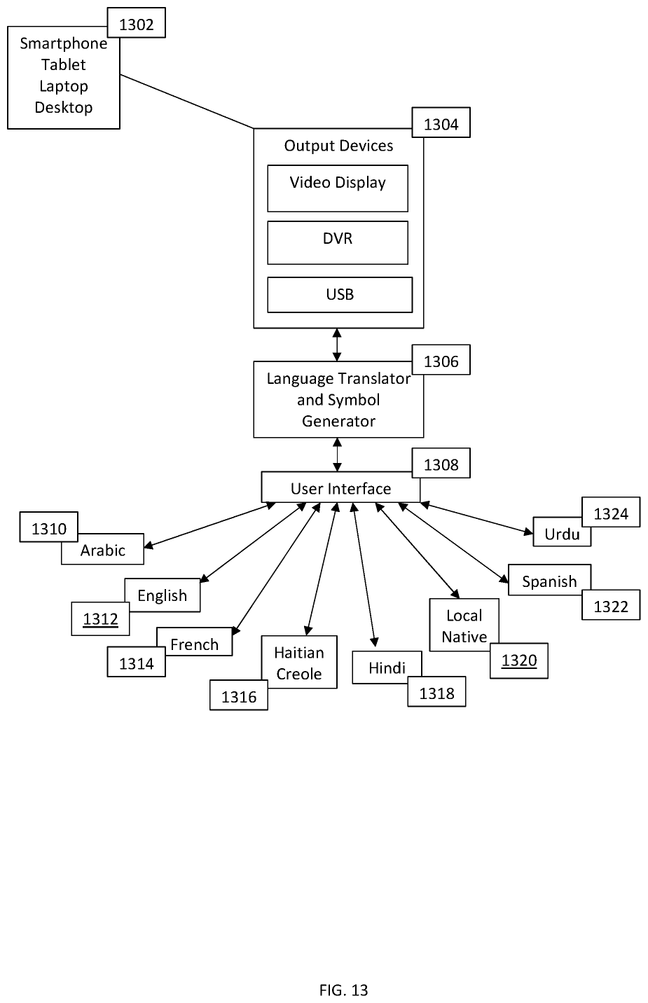

FIG. 13 illustrates how the software system of the present invention can be adapted for uptake in various host countries. As described above, local acceptance is a key initial hurdle to overcome and the first thing that has to happen is that the system be tailored to the local prevalent language. A user in a given country will interact with the system via a means 1302 that can be a smartphone, tablet, laptop or desktop computing means, and made visually accessible via a video display, digital video recording device means or universal serial bus (USB)-enabled port in a particular device 1304. The system has a language translator and symbol generator module 1306 that will configure all outputs into a language prevalent in the locale, and will generate symbols wherever possible to communicate messages or instructions. An exemplary non-limiting list of languages that may predominate in a developing country includes in alphabetical order, but is not limited to, Arabic 1310, English 1312, French 1314, Haitian Creole 1316, Hindi 1318, a local dialect or less prevalent language 1320, Spanish 1322, or Urdu 1324.

Each of the modules of the software system of the present invention has been presented with descriptions of the manner in which they execute their respective programs to address the factors used to assess and improve a developing nation's health system.

Use of the system and its modules will result in ever-expanding database. This ever-expanding database of national medical and healthcare, however, is additionally drawn upon in parallel by all other modules in the software system of the invention to carry out their respective functions and further their respective factors that are used to measure, and thereby achieve, progress toward the improvement of the country's national healthcare system.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice well known to those of ordinary in the art or arts upon which this invention relies, and to which this invention pertains.

What is claimed is:

1. A national healthcare data management tool system adapted for execution on a computer system coupled to a global computer network, in an architecture of a server in communication with a plurality of clients in a network, comprising:
(a) at least one predictive artificial intelligence module for processing medical or health data received from one or more specialized computation modules, in consideration of patient data to identify a diagnosis and therapy for a patient, or in consideration of patient data from a population of patients to identify therapy for a diagnosis common to members of the patient population;
(b) a national medical information database;
(c) a gateway module configured to provide said artificial intelligence module with access to said database; and
(d) a communications module operable to enable any of said modules to send or receive data from any other module in said healthcare management tool system, and to send data to, or receive data from, a plurality of clients.

2. The system as claimed in claim 1, additionally comprising an education module operable to present medical educational content to one or more clients in said network.

3. The system as claimed in claim 1, additionally comprising a medical provider evaluation module operable to present an evaluation of appropriateness of a medical provider's diagnosis or treatment decisions and communicate recommendations to said provider in consideration of said evaluation of appropriateness.

4. The system as claimed in claim 1, additionally comprising a communicable disease module operable to receive communicable disease data from one or more of said network clients, present said data to said artificial intelligence module, receive output data from said artificial intelligence module, and send output data on said communicable disease to one or more of said clients.

5. The system as claimed in claim 1, additionally comprising a specialist referral module operable to assess a need for the participation of a specialty physician in the management of a patient's case and assist in the location of such a specialty physician, and assist in coordinating a patient examination by said specialty physician.

6. The system as claimed in claim 1, additionally comprising a medical device data transfer module operable to receive data from one or more medical devices capable of digitizing medical device output data, and to present said medical device output data to said artificial intelligence module for processing in the diagnosis and treatment of a patient.

7. The system as claimed in claim 1, additionally comprising a medicines supply module operable to automatically query for, receive, and organize inventory information for data collection and analysis in locating and reporting amounts and locations of medicines or medical supplies required by a client in said network.

8. The system as claimed in claim 1, additionally comprising a disaster relief coordination module for receiving disaster-related data from one or more of said network clients at the location of a disaster, and operable to automatically query for, receive, locate, coordinate, and communicate disaster relief personnel or supplies for dispatch to the location of a disaster.

9. The system as claimed in claim 1, additionally comprising a national health delivery module operable as a data presentation module for presenting healthcare recommended courses of action to be implemented across a national healthcare system.

10. The system as claimed in claim 1, additionally comprising a public health key indicator module for addressing identified measures of public health across a national public health system with recommended actions calculated to achieve improvement in said measures with maximal economic effectiveness and shortest time effectiveness.

11. The system as claimed in claim 1, additionally comprising a global health grant application module operable to coordinate and present healthcare data from any of said modules in a format suitable for review of an economic assistance grant application by a public health economic grant agency or philanthropic organization.

12. A national healthcare data management tool system adapted for execution on a computer system coupled to a global computer network, in an architecture of a server and a plurality of clients in a network, comprising:
(a) at least one predictive artificial intelligence module for processing medical or health data received from one or more specialized computation modules, in consideration of patient data to identify a diagnosis and therapy for a patient, or in consideration of patient data from a population of patients to identify therapy for a diagnosis common to members of the patient population, said artificial intelligence module in communication with a suitable computer server means to enable input and data processing from, and presenting output to, said specialized computation modules or said clients, said number of specialized computation modules selected from the group consisting of:
(b) an education module operable to present medical educational content to a client;
(c) a medical provider evaluation module operable to present an evaluation of appropriateness of a medical provider's diagnosis or treatment decisions;
(d) a communicable disease module operable to receive communicable disease data from one or more of said clients, present said data to said artificial intelligence module, receive data from said artificial intelligence module, and send data to one or more of said clients;
(e) a specialist referral module operable to assess a need for the participation of a specialty physician in the management of a patient's case and assist in the location of such a specialty physician;
(f) a medical device data transfer module operable to receive data from a plurality of medical devices and present said medical device data to said artificial intelligence module for processing;
(g) a medicines supply module operable to automatically query for, receive, and organize inventory information for data collection and analysis in locating and reporting amounts and locations of medicines or medical supplies; and
(h) a communications module operable to enable any of said modules to send or receive data from any other module in said healthcare management tool system, and to send data to, or receive data from, a plurality of clients.

13. The system as claimed in claim 12, additionally comprising a national medical information database, and a gateway module configured to provide said artificial intelligence module with access to said knowledgebase.

14. The system as claimed in claim 12, additionally comprising a disaster relief coordination module for receiving disaster-related data from one or more of said clients, and locating and coordinating disaster relief personnel or supplies for dispatch to the location of a disaster.

15. The system as claimed in claim 12, additionally comprising a national health delivery module operable as a data presentation module for presenting healthcare recommended courses of action to be implemented across a national healthcare system.

16. The system as claimed in claim 12, additionally comprising a public health key indicator module for addressing identified measures of public health across a national public health system with recommended actions calculated to achieve improvement in said measures with maximal economic efficiency.

17. The system as claimed in claim 12, additionally comprising a global health grant application module operable to coordinate and present healthcare data from any of said modules in a format suitable for review by a public health economic grant agency or philanthropic organization.

18. A national healthcare data management tool method, comprising the steps of:
(a) receiving and storing medical and healthcare information from a physician or a physician's support staff;
(b) identifying a diagnosis and one or more treatment recommendations according to the medical and healthcare information received;
(c) storing and organizing the diagnostic and treatment recommendations; and
(d) retrieving the organized diagnostic and treatment recommendations and processing the diagnostic and treatment for the creation of a national healthcare database.

19. The method as claimed in claim 18, where the created national healthcare database is applied to one or more national healthcare purposes selected from the group consisting of:
(a) educating medical and healthcare personnel;
(b) evaluating the appropriateness of a medical or healthcare worker's individual patient diagnosis and treatment strategy;
(c) managing the outbreak of a communicable disease across a national population;
(d) referring an individual patient to a physician in a medical specialty;
(e) applying digitized data from one or more medical devices to the diagnosis and treatment of a patient;
(f) organizing information on the availability of medicines or medical supplies within a national healthcare system;
(g) coordinating disaster relief;
(h) recommending courses of action to be applied across a national healthcare system for improvement of the system's healthcare delivery;
(i) identifying and efficiently addressing factors used to measure the effectiveness of a national healthcare system; or
(j) applying for healthcare funding from funding agencies or philanthropic organizations.
